(19) United States
(12) Patent Application Publication
(10) Pub. No.: US 2005/0113292 A1
(43) Pub. Date: May 26, 2005

(54) Compositions of protein mimetics and methods of using same against HIV-1, SARS-coV and the like

(75) Inventors: James P. Tam, Nashville, TN (US); Qitao Yu, Nashville, TN (US); Yi-An Lu, Nashville, TN (US); Jin-Long Yang, Nashville, TN (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Vanderbilt University, Nashville, TN (US)

(21) Appl. No.: 10/893,551

(22) Filed: Jul. 16, 2004

Related U.S. Application Data
(60) Provisional application No. 60/488,558, filed on Jul. 18, 2003.

(51) Int. Cl. A61K 38/16; C07K 14/16

(52) U.S. Cl. 514/8; 530/395

(57) ABSTRACT

The present invention provides compositions of protein mimetics and methods of using same against HIV-1, SARS-coV and the like. In one aspect, the present invention relates to a protein mimetic for preventing HIV-1 entry to host cells of a living subject through membrane fusion, wherein HIV-1 contains at least one envelope glycoprotein gp41 that has a plurality of peptides in a pre-hairpin state. In one embodiment, the protein mimetic comprises at least two monomeric peptide strands and an interstrand linker coupling the monomeric peptide strands. The coupled monomeric peptide strands prevent the plurality of trimeric gp41 in a pre-hairpin state from transiting to a six-helix hairpin bundle, thereby inhibiting HIV-1 entry to the host cells through membrane fusion.

### CROSS-REFERENCE TO RELATED PATENT APPLICATION

This application claims the benefit, pursuant to 35 U.S.C. § 119(e), of provisional U.S. patent application Ser. No. 60/488,558, filed Jul. 18, 2003, entitled “COMPOSITIONS AND METHODS FOR THE USE OF PROTEIN FUSION INHIBITORS AGAINST HIV-1, SARS-COV AND THE LIKE,” by James P. Tam, Qitao Yu, Yi-An Lu, and Jin-Long Yang, which is incorporated herein by reference in its entirety. Some references, which may include patents, patent applications and various publications, are cited in a reference list and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [68] represents the 68th reference cited in the reference list, namely, Tam JP, Lu Y-A, Yang J-L, Chiu K-W, An unusual structural motif of antimicrobial peptides containing end-to-end macrocycle and cystine-knot disulfides. Proc. Natl. Acad. Sci. USA. 96:8913-8918. 1999.

This invention was made with government support under a grant NIH AI46164 awarded by the National Institute of Health. The government has certain rights in the invention.

### FIELD OF THE INVENTION

The present invention generally relates to protein mimetics, more specifically, to compositions of protein mimetics and methods of using same against HIV-1, SARS-coV and the like.

### BACKGROUND OF THE INVENTION

The human immunodeficiency virus (HIV) is a pathogenic retrovirus and the causative agent of acquired immune deficiency syndrome (AIDS) and related disorders [89,90]. There are at least two distinct types of HIV: HIV-1 [89,90] and HIV-2 [91,92]. Virtually all AIDS cases in the United States are associated with HIV-1 infection. A large amount of genetic heterogeneity exists within populations of each viral type.

HIV is a member of lentivirus family of retroviruses [116]. Retroviruses are small-enveloped viruses that contain a diploid, single-stranded RNA genome, and replicate via a DNA intermediate produced by a virally encoded reverse transcriptase, an RNA-dependent DNA polymerase [93]. The HIV viral particle has a viral core, made up of proteins designated p24 and p18. The viral core contains viral RNA genome and enzymes required for replicative events. Myristylated gag protein forms an outer viral shell around the viral core, which is in turn surrounded by a lipid membrane envelope derived from the infected cell membrane. The HIV envelope surface glycoproteins are synthesized as a single 160 kD precursor protein, which is cleaved by a cellular protease during viral budding into two glycoproteins, gp41 and gp120, respectively. gp41 is a transmembrane protein and gp120 is an extracellular protein that remains noncovalently associated with gp41, possibly in a trimeric or multimeric form [94].

HIV targets CD-4+ T lymphocytes because the CD-4 surface protein acts as cellular receptor for the virus [95,96]. Viral entry into cells is dependent upon gp120 binding to the cellular CD-4 receptor while gp41 anchoring the envelope glycoprotein complex in the host cell membrane [96,97], thus explains HIV's tropism for CD-4+ cells. Infection of human CD-4+ T-lymphocytes with HIV leads to depletion of the cell type and eventually to opportunistic infections, neurological dysfunctions, neoplastic growth, and untimely death.

HIV infection is pandemic and HIV associated diseases represent a major world health threat. Although considerable effort is being put into the successful design of effective therapeutics, currently no curative anti-retroviral drugs against AIDS exist to the best knowledge of the inventors. In attempts to develop such drugs, almost every stage of the viral life cycle has been considered as target for therapeutic intervention [98].

Virally encoded reverse transcriptase targeted drugs, including 2′,3′-dideoxynucleoside analogs such as AZT, ddI, ddC, and d4T, have been shown to be active against HIV [99]. While beneficial these nucleoside analogs are not curative, probably due to the rapid appearance of drug resistant HIV mutant strains [100]. In addition, the drugs often exhibit toxic side effects such as bone marrow suppression, vomiting, and liver function abnormalities.

Late stages of HIV replication, which involve crucial virus specific secondary processing of certain viral proteins have also been suggested as possible anti-HIV drug targets. Late stage processing is dependent on the activity of a viral protease, and drugs are being developed to inhibit this protease [101]. The clinical outcome of these candidate drugs may still be in question.

Attention is also being given to the development of vaccines for the treatment of HIV infection. The HIV-1 envelope proteins (gp160, gp120, gp41) have been shown to be the major antigens for anti-HIV antibodies present in AIDS patients [102]. Thus far, these proteins seem to be the most promising candidates to act as antigens for anti-HIV development. To this end, several groups have begun to use various portions of gp160, gp120, and/or gp41 as immunogenic targets for the host immune systems. See for example, Ivanoff, L. et al., U.S. Pat. No. 5,141,867; Saith, G. et al., WO 92/22, 654; Schafferman, A., WO 91/09,872; Formoso, C. et al., WO 90/07,119. Clinical results concerning these candidate vaccines are forth coming.

Recently, double stranded RNAs, which elicit a general immune response; have been used in combination with antivirals such as interferon, AZT and phosphonoformate to treat viral infections. See Carter, W., U.S. Pat. No. 4,950,652. In addition, a therapy combining a pyrimidine nucleoside analog and a uridine phosphorylase inhibitor has been developed for the treatment of HIV, see Sommadossi, J. P. et al., U.S. Pat. No. 5,077,280. Although these specific therapies may prove to be beneficial, combination therapy in general has the potential for antagonism as demonstrated in vitro with AZT and ribavirin. See U.S. Pat. No. 4,950,652. Moreover, combination therapy is potentially problematic given the high toxicity of most anti-HIV therapeutics and their low level of effectiveness.

HIV entry and fusion stages of the infection also offer many opportunities for intervention. Indeed, modalities to inhibit virtually every step in this pathway using small molecules to proteins are being vigorously explored [8-15]. These include inhibitors against CD-4 attachment, chemokine coreceptor, and membrane fusion [16-20].

The focus of viral entry has been on CD-4, the cell surface receptor for HIV. For example, recombinant soluble CD-4 has been shown to block HIV infectivity by binding to viral particles before they encounter CD-4 molecules embedded in cell membranes [103]. Certain primary HIV-1 isolates are relatively less sensitive to inhibition by recombinant CD-4 [104]. In addition, recombinant soluble CD-4 clinical trials have produced inconclusive results [104,105].

Membrane fusion of HIV is mediated by two noncovalently associated subunits of HIV envelope glycoprotein, gp120 and gp41. HIV gp120 directs target-cell recognition and viral tropism through interaction with the cell-surface receptor CD-4 and a chemokine coreceptor [1-3]. The membrane anchored gp41 then promotes fusion of the viral and cellular membranes, resulting in the release of viral contents into the host cell [4-10].

A pre-hairpin model, which is further described infra in connection with FIG. 1, depicting the interplay between gp120 and gp41 in the HIV fusion events has been proposed based on snapshots of their tertiary soluble structures, precedents of fusion proteins from other viral families, and biochemical studies by many laboratories [4-10]. Several aspects of the pre-hairpin model are as follows: (1) constitutively expressed native (non-fuseogenic) state contains noncovalent complex of gp 120 and gp41 in trimeric forms and part of gp41 is masked by gp120; (2) fusion active state contains a pre-hairpin intermediate after gp120 binding to CD-4 and a chemokine coreceptor that results in conformation changes to expose gp41 and insertion into the target cell membrane, making gp41 an integral protein in two different membranes; (3) after dissociation from gp120 and conformational changes, the pre-hairpin intermediate of gp41 forms a stable six-helix bundle that results in fusion of two membranes and viral entry into host cells. The primary sequence of gp41 contains two heptad-(sequence of seven amino acids) repeating regions predictive of helical structures. The six-helix bundle (trimers-of-hairpin) is formed by three heptad-repeats in region 1 (HR1) at the amino terminus (N-peptides) as a coiled-coil structure and the heptad repeats in region 2 (HR2) at the carboxyl terminus (C-peptides) that folds back like a hairpin into the hydrophobic grooves of the coiled-coil. The heptad repeats contain hydrophobic amino acids at positions 1 and 4 of the heptad.

The gp41 fusion intermediates contain multiple epitopes that are transiently exposed during fusion and can provide targets for therapeutic intervention. Binders of gp41 fusion intermediate are thought to act as dominant negative inhibitors that prevent the transition from the pre-hairpin intermediate to the six-helix bundle, the fusion-state driving force for integrating the viral and target cell membranes. Several agents have been identified that block HIV-1 infection by targeting gp41 fusion intermediates. These agents include the gp41-based peptides T-20 (formerly known as DP178, a 36-residue C-peptide sold under the trade name Fuzeon), T-1249, DP107, N34, C28, and various fusion proteins and analogues thereof [106-112]. Other studies have identified inhibitors that comprise non-natural D-peptides and nonpeptidyl moieties [113,114]. Clinical proof-of-concept for this class of inhibitors has been provided by T20, which reduced plasma HIV RNA levels by as much as 2 logs in Phase I/II human clinical testing [115]. The broad antiviral activity demonstrated for this class of inhibitors reflects the high degree of gp41 sequence conservation amongst diverse strains of HIV-1.

The clinical success and limitations of T20 as a therapeutic have led to the desire to identify small molecules, particularly an orally bioavailable molecule mimicking the function of C-peptides. Most approaches utilize the X-ray crystal structure of the gp41, including the use of computational methods and molecular docking techniques as well as high throughput methods using combinatorial libraries and phage display to screen and test organic compounds, small peptides and peptidomimetics [32-35]. Unlike the effort in developing HIV post-entry inhibitors such as those against reverse transcriptase and protease, small-molecule approaches have not yielded, at this time, specific compounds with potency and specificity that surpasses T20 or its analogs. These efforts are still ongoing in many laboratories and it is too early to arrive at a conclusion. However, it is likely that the binding surface and mechanism to inhibit the pre-hairpin intermediate may be different from the traditional receptor-ligand complex that has been so successfully exploited for developing enzyme inhibitors.

Thus far, T20 (DP178) is the only approved representative. In clinical trials, T20 is as effective as the HAART regimen in reducing viral load [24]. Early report estimates the cost for a yearly regimen of T20 at $20,000. Its high price is in part due to its high production cost and high-dose regimen at 100 to 200 mg/day. Such a high cost poses an impediment to wide affordability to AIDS patients. Another concern is that T20 resistance in clinical trials has emerged and a more potent fusion inhibitor may reduce such risk [29-31].

Therefore, a heretofore unaddressed need exists in the art to address the aforementioned deficiencies and inadequacies.

### SUMMARY OF THE INVENTION

In one aspect, the present invention relates to a protein mimetic for preventing HIV-1 entry to host cells of a living subject through membrane fusion, wherein HIV-1 contains at least one envelope glycoprotein gp41 that has a plurality of peptides in a pre-hairpin state. In one embodiment, the protein mimetic comprises at least two monomeric peptide strands and an interstrand linker coupling the monomeric peptide strands. The coupled monomeric peptide strands prevent the plurality of trimeric gp41 in a pre-hairpin state from transiting to a six-helix hairpin bundle, thereby inhibiting HIV-1 entry to the host cells through membrane fusion. Each of the two peptide strands has an amino acid sequence, which contains at least one of N36, DP178, T1249, C34, any other amino acid sequences derived from N-peptide or C-peptide regions of gp41, or any truncated, mutated, modified linear or cyclized analogs thereof. Moreover, the at least two monomeric peptide strands can be the same or chimeric. The at least two monomeric peptide strands can be coupled by the interstrand linker through a chemical, enzymatic, or biological synthetic method. The chemical synthetic methods include but not limited to chemoselective thiazolidine ligation, Trp-ligation, ψGly ligation, Michael addition ligation, disulfide linkage, or any combination thereof. In one embodiment, the interstrand linker has at least two arms represented by formula 1 or 2: 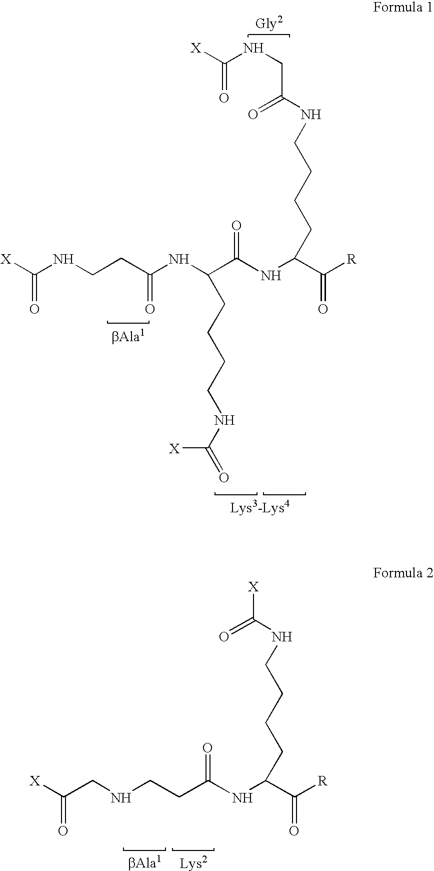
wherein X can be an aldehyde, β-aminoethyl thiol, chloroacetyl or acrylate, and R is Ser-Ser-Ala-NH₂.

In another aspect, the present invention relates to a pharmaceutical composition suitable for administration to a living subject for preventing or treating infections caused by HIV-1 viral entry to host cells of the living subject through membrane fusion, wherein HIV-1 contains at least one envelope glycoprotein gp41 that has a plurality of peptides in a pre-hairpin state. In one embodiment, the pharmaceutical composition has a pharmaceutically acceptable protein mimetic having at least two monomeric peptide strands and an interstrand linker coupling the monomeric peptide strands. The pharmaceutical composition further has a pharmaceutically acceptable carrier suitable for administration to a living subject.

In yet another aspect, the present invention relates to a therapeutic or prophylactic method against HIV-1 infection by inhibiting viral entry to host cells of a living subject through membrane fusion, wherein HIV-1 contains at least one envelope glycoprotein gp41 that has a plurality of peptides in a pre-hairpin state. In one embodiment, the therapeutic or prophylactic method includes the step of administering to a living subject an effective amount of a protein mimetic, wherein the protein mimetic has at least two monomeric peptide strands and an interstrand linker coupling the monomeric peptide strands.

In a further aspect, the present invention relates to a protein mimetic for preventing viral entry of a virus to host cells of a living subject through membrane fusion, wherein the virus contains at least one protein that has a plurality of peptides in a pre-hairpin state. In one embodiment, the protein mimetic has at least two monomeric peptide strands and an interstrand linker coupling the monomeric peptide strands. The coupled monomeric peptide strands prevent the plurality of peptides of the protein in a pre-hairpin state from transiting to a hairpin bundle, thereby inhibiting viral entry of the virus to the host cells through membrane fusion. The virus can be one of HIV-1, Ebola, influenza, SARS-coV, retroviruses, corona viruses, orthomyxoviruses or paramyxoviruses. Moreover, each of the two strands has an amino acid sequence that is derived from N-peptide or C-peptide regions of the protein, or any truncated, mutated, modified linear or cyclic analogs thereof.

In another aspect, the present invention relates to a pharmaceutical composition suitable for administration to a living subject for preventing or treating infections caused by viral entry of a virus to host cells of the living subject through membrane fusion, wherein the virus contains at least one protein that has a plurality of peptides in a pre-hairpin state. In one embodiment, the pharmaceutical composition includes a pharmaceutically acceptable protein mimetic that has at least two monomeric peptide strands and an interstrand linker coupling the monomeric peptide strands. The pharmaceutical composition further has a pharmaceutically acceptable carrier suitable for administration to a living subject.

In yet another aspect, the present invention relates to a therapeutic or prophylactic method against viral infection by inhibiting viral entry of a virus to host cells of a living subject through membrane fusion, wherein the virus contains at least one protein that has a plurality of peptides in a pre-hairpin state, In one embodiment, the therapeutic or prophylactic method includes the step of administering to a living subject an effective amount of a protein mimetic, wherein the protein mimetic has at least two monomeric peptide strands and an interstrand linker coupling the monomeric peptide strands.

In a further aspect, the present invention relates to a protein mimetic for inhibiting membrane fusion, wherein the membrane contains at least one protein that has a plurality of peptides in a pre-hairpin state. In one embodiment, the protein mimetic has at least two monomeric peptide strands and an interstrand linker coupling the monomeric peptide strands. In one embodiment, the coupled monomeric peptide strands prevent the plurality of peptides of the protein in a pre-hairpin state from transiting to a hairpin bundle, thereby inhibiting membrane fusion. The membrane fusion can be vesicle fusion or any membrane fusion event that involves a hairpin-mediated step. Moreover, each of the two strands has an amino acid sequence that is derived from N-peptide or C-peptide regions of the protein, or any truncated, mutated, modified linear or cyclic analogs thereof.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 depicts a pre-hairpin model of HIV and host cell fusion cascade.

FIG. 2 shows a schematic diagram of gp41 and related peptides.

FIG. 3 shows CD (circular dichroism) spectra of 3α-DP178 and DP-178 (panel A), 3α-C34 and C34 (panel B) as well as the thermal stability of 2α- and 3α-DP178 (panel C), respectively.

FIG. 4 depicts peptide mixing experiments that show antagonistic inhibition of R9 HIV-1 virus infection of T20 (panel A), 2α- (panel B) and 3α-T20 (panel C) with 3α-N36 and CD spectra of 3α-T20 before and after mixing with N36 (panel D), respectively.

FIG. 5 depicts neutralization of gp41 trimer peptide immune sera for T-tropic HIV-1 R8 (panel A) and neutralization of gp41 trimer peptide immune sera for M-tropic HIV-1 HIV-1BAL (panel B), respectively.

FIG. 6 depicts interstrand linkers IL-1 a-c, IL-2 a-c and IL-3 a-c with attached functional groups (a=aldehyde, b=β-aminoethyl thiol or chloroacetyl, c=acrylate).

FIG. 7 depicts Thz- and Trp-ligation of 2α and 3α mimetics.

FIG. 8 depicts ψGly ligation to prepare 2α and 3α mimetics.

FIG. 9 depicts Michael addition to prepare 2α and 3α mimetics with interstrand linker at C-terminus.

FIG. 10 depicts global modification of solvent accessible surface of C34 peptides with intrahelical salt bridges.

FIG. 11 depicts Thz- and Trp-ligation to prepare 3α double constraint protein mimetics.

FIG. 12 depicts tandem Cys-cyclization and Michael addition to prepare cyclic peptide protein mimetics.

FIG. 13 depicts stability test of T20 and 3α-T20 against proteolytic digestion, respectively.

FIG. 14 depicts the structures of 3α-C34 and 3α-T20 with different interstrand linkers.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “living subject” refers to a human being such as a patient, or an animal such as a lab testing monkey.

As used herein, “3α-N36” or “3α-C34” mimetic refers to the 3-helix trimer of N36 (a 36-residue N-peptide of HR1) or C-34 (a 34-residue C-peptide of HR2), respectively. To denote their design and form, placing 3α on the left of N36 or C34 indicates that an interstrand linkage is located at the amino terminus according to the conventional nomenclature of proteins. For carboxyl-linked mimetics such as N36-3α and C34-3α mimetics, 3α is placed at the right to signal an interstrand linkage at the carboxyl terminus. Similar notations are used for the 2-helix dimers, 2α-mimetics with two interlinked strands. For double-constrained 2- or 3-helix mimetics with interstrand linkers placed at both termini, they are denoted for example as “2α-[C34]” or “3α-[C34]” for the C34 mimetics.

As used herein, the following standard abbreviations are used throughout the specification to indicate specific amino acids: A=Ala=alanine R=Arg=arginine N=Asn=asparagine D=Asp=aspartic acid C=Cys=cysteine Q=Gln=glutamine E=Glu=glutamic acid G=Gly=glycine H=His=histidine I=Ile=isoleucine L=Leu=leucine K=Lys=lysine M=Met=methionine F=Phe=phenylalanine P=Pro=proline S=Ser=serine T=Thr=threonine W=Trp=tryptophan Y=Tyr=tyrosine V=Val=valine.

As used herein, “epitope” means a portion of a molecule or molecules that form a surface for binding antibodies or other compounds. The epitope may comprise contiguous or noncontiguous amino acids, carbohydrate or other nonpeptidyl moieties or oligomer-specific surfaces.

As used herein, “T20” and “DP178” are used interchangeably.

As used herein, “administering” may be effected or performed by using any of the methods known to one skilled in the art, which includes intralesional, intraperitoneal, intramuscular, subcutaneous, intravenous, liposome mediated delivery, transmucosal, intestinal, topical, nasal, oral, anal, ocular or otic delivery. The compounds may be administered together or separately (e.g., by different routes of administration, sites of injection, or dosing schedules) so as to combine in synergistically effective amounts in the subject.

### OVERVIEW OF THE INVENTION

Among other things, applicants have invented substance protein mimetic, which has at least two monomeric peptide strands and an interstrand linker coupling the monomeric peptide strands and methods of using same as membrane fusion inhibitor. In a particular example, the protein mimetic can be used in a variety of applications including a pharmaceutical composition or a therapeutic or prophylactic method against HIV-1 infection.

The inventors use protein mimetic as second generation pre-entry HIV inhibitors. While peptide mimetics generally mimic the bioactive conformation of molecules at the secondary-structure level, protein mimetics concerns mimicry at the protein level involving tertiary structures. One advantage of using protein mimetics is that it better duplicates the structure of the target protein than peptide mimetics. Another difference between peptide and protein mimetics is stability. Because they are helical with both hydrophobic and hydrophilic faces, peptide mimetics are also prone to aggregation. In contrast, protein mimetics allows internal stabilization of their hydrophobic faces and exposing only their hydrophilic faces to solvents that increase solubility and minimize aggregation.

Because of the sequence variability of HIV-1 envelope proteins, the composition, size and precise location of such sequences may be different for different viral isolates. The gp41 fusion intermediates may also present other linear or conformational epitopes that are transiently expressed during HIV-1 entry. An inhibitor may target multiple epitopes present on gp41 fusion intermediates. Alternatively, separate inhibitors may be used in combination to target one or more epitopes present on gp41 fusion intermediates.

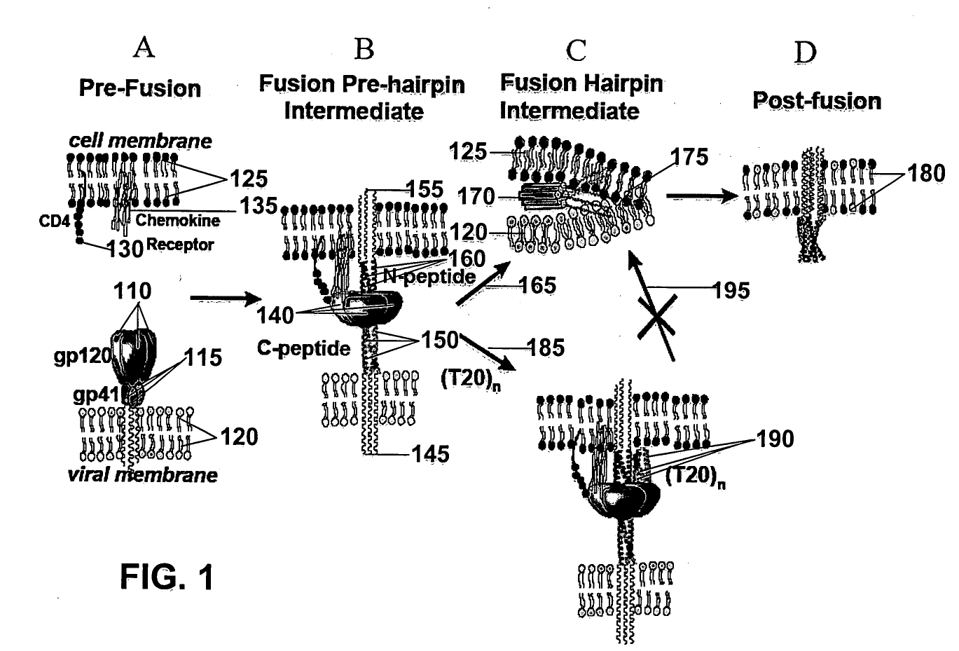

Infection by HIV-1 requires fusion of the viral and target cell membranes mediated by viral envelope glycoprotein gp120 and gp41. This process offers opportunities for intervention because 3-dimensional structures of critical proteins involved have been determined, including gp41, which forms trimers of hairpins commonly involved in the final step of membrane fusion. A promising target therefore is a fusion active pre-hairpin intermediate of gp41 that is exposed after gp120 binding with cell surface receptors. In this fuseogenic state, the pre-hairpin cross links two different membranes, exposing an amino- and an carboxyl-homotrimeric α-helical coiled ectodomains that eventually form a six-helix bundle hairpin bringing the amino- and the carboxyl-terminal regions of the gp41 ectodomains into close proximity enabling membrane fusion. More specifically as illustrated in FIG. 1, the entire fusion process may be divided into four continuous stages. In pre-fusion stage A, noncovalent complex of gp120 (110) and gp41 (115) are constitutively expressed on viral membrane 120 as trimers in native state, while CD-4 (130) and chemokine coreceptor (135) are present on the target cell membrane 125 as potential viral receptors. In fusion pre-hairpin intermediate stage B, when binding to CD-4 and chemokine coreceptor, gp120 undergoes a conformational change 140 that exposes gp41, which is transformed to a pre-hairpin intermediate. This intermediate of gp41 has three homotrimeric α-helical coiled-coil strands with the carboxyl terminal 145, which is composed of three identical C-peptides 150 planted in viral membrane 120 and with the amino terminal 155, which is composed of three identical N-peptides 160 inserted into the target cell membrane, making gp41 an integral protein in two different membranes.

The N-peptide portion of gp41 has three heptad repeat regions called HR1 in which heptad means sequence of seven amino acids and in this case contains hydrophobic amino acids at position 1 and 4 of the heptad sequence. Three HR1s of the N-peptides can self-assemble to a coiled-coil structure that is characterized by a hydrophobic groove. The C-peptide portion of gp41 has heptad repeats referred to as HR2. When there is no external interference 165, the three HR2s of the C-peptides fold back like a hairpin into the hydrophobic grooves of the HR1 coiled-coil to form a 6-helix bundle 170. In doing so, the process advances to fusion hairpin intermediate stage C. The initial partially fused membranes 175 of the virus 120 and the target cell 125 continue the fusion until the process advances to the post-fusion stage D where two membranes are completely fused together 180. If T20 (190) is present in the process, it competes with 185 the C-peptides to bind to the N-peptides of the intermediate, causing the fusion process to stop at stage C, where membrane fusion did not and will not occur 195.

Similar to T20, synthetic peptides targeting N- or C-peptide domains are therefore, effective fusion inhibitors and constitute a new class of HIV pre-entry therapeutics. However, the first-generation peptidyl drug candidates have limitations that include high dosage and poor stability. The present application focuses on developing novel protein mimetics of gp41 as second-generation fusion inhibitors to improve the potency and stability of the peptide-based therapeutics.

The fusion inhibiting synthetic peptide as a single amphipathic coil is structurally unstable and prone to aggregation and proteolytic degradation and therefore, lacks the advantages offered by the trimeric coil found in the pre-hairpin.

To enhance their bioactive conformation and stability, synthetic peptides can be covalent linked to form a trimeric coiled-coil, called 3α mimetics as covalent-linked trimeric coiled-coils can confer stable structures and better mimics of the fuseogenic conformation. Engineered constructs of N-peptides as proteins have been designed to target specifically the C-region in the pre-hairpin intermediate. They include the chimeric protein NccG-gp41, which features an exposed disulfide-linked trimeric N-helices grafted onto an ectodomain of gp41 [36]; peptides in which the trimeric protein is stabilized by fusion to the GCN4 trimeric coiled-coil [23]; and the protein 5-helix, in which the internal trimer coiled-coil of N-helices is surrounded by only two C-helices [24]. These engineered proteins show a significant increase in potency when compared with the corresponding N-peptides, but are less potent than T20. Nevertheless, they provide support for 3a protein mimetics design principle and suggest that a stable mimetics of a trimeric version of either HR1 (N-peptide) or HR2 (C-peptide) may increase potency. Surprisingly, an engineered construct of a trimeric version of T20 or other HR2 region has not been reported. This is in part due to the influence of the pre-hairpin model. Engineering N-peptides as a trimeric coiled-coil is logical because the trimeric N-peptides form a trimer coiled-coil that is the core of the six-helix bundle of the hairpin fusion protein. In contrast, the C-peptides fold back to the N-peptide trimer in an antiparallel fashion against a groove created by the N-peptide trimer. Nevertheless, this model implies that the bioactive conformation of the C-peptide is helical and may exist as a helical trimeric intermediate in a certain pre-hairpin state. Towards this end, the applicants have designed 3-helix (3α) mimetics with covalent-linked trimeric C- and N-peptides.

A 5-helix bundle containing five of the six helices that make up the core of the gp41 trimer-of-hairpins structures, lacking a third C-peptide is engineered [15]. The 5-helix protein is aqueous stable, serves as a high-affinity binding site for the C-helix of gp41 and displays IC₅₀ at low nano molar concentrations. Consequently, protein mimetics containing dimeric and parallel-coiled C-peptides might bind to the N-helix of gp41 to form a 5-helix bundle. As such, the 2α mimetics of C-peptides are expected to be more potent than the 5-helix bundle because C-peptides are generally more potent than N-peptides, the intended target of 5-helix bundle. Further, 2α mimetics is significantly smaller than the 5-helix bundle and simpler for chemical synthesis than 3α mimetics.

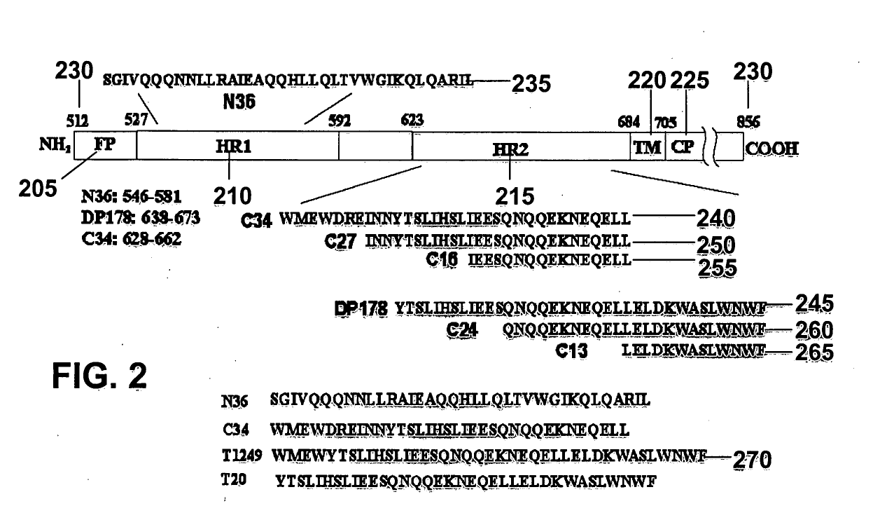

As disclosed above, the 2α and 3α mimetics are intended to mimic the bioactive conformation of the gp41 pre-hairpin while retaining a protein-like structure, which is achieved by constraining two or three monomeric peptides with an interstrand linker as dimers and trimers to overcome the energy barrier of oligomerizing into coiled-coils. The linker can be placed at either N- or C-, or both terminus of the peptides. The monomeric peptides used can be identical or chimeric, linear or cyclic. The amino acid sequences of monomeric peptides are derived from the HR1 (N-peptide) or HR2 (C-peptide) region of gp41. As illustrated in FIG. 2, important functional regions of gp41 include fusion peptide (FP, 205), two heptad-repeat regions HR1 (210) and HR2 (215), the transmembrane region (TM, 220) and the cytoplasmic domain (CP, 225). Small numbers on top of the diagram 230 indicate amino acid numbering in the sequence of gp41. N36 is derived from the HR1 N-peptide region 235 and is composed of amino acid sequence 546-581. C34 is derived from the HR2 C-peptide region 240 and is composed of amino acid sequence 628-662. DP178 (T20) is also derived from the HR2 C-peptide region 245 and is composed of amino acid sequence 638-673. Selected truncated analogs 250, 255, 260, 265, and 270 are derived from potent C-peptides DPI78 and C34, correspond to C27, C16, C24, C13, and T1249 respectively. The unprotected peptides DP178, C34, N36 or their truncated analogs prepared by solid-phase synthesis are tethered to an interstrand linker that has two or three flexible arms. For example, a Cys is placed in their N-terminus and thiazolidine (Thz-) ligation is used to link them to an aldehyde functionalized linker.

The multimeric and parallel strands of the proposed 2α and 3α mimetics are artificial proteins that pose a synthetic challenge by recombinant methods. The 3α mimetics are three-stranded compounds, containing three pairs of carboxylic and amino termini. Biosynthetic preparation of such mimetics with two or three interlinked parallel helical strands is formidable because stably folded 2α or 3α proteins will have to contain at least one anti-parallel strand. Indeed, previous work by recombinant methods produced either five- or six-helix bundles containing both N- and C-peptides in an up-and-down fashion similar to the fusion hairpin [20]. Chemoselective ligation is well suited to couple the monomeric peptides with a linker without an anti-parallel strand. Experimentally, N- or C-peptides such as the T20 monomer can be ligated using ligation methods that the applicant's laboratory has developed over the past decade [38-50] to chemoselectively form 2α and 3α mimetics in aqueous solutions without a coupling reagent. This method maximizes flexibility and minimizes chemical steps to afford various molecules for different experimental needs.

Chemoselective ligation exploits the mutual reactivity of a pair of nucleophile and electrophile. In general, a nucleophile is placed at the N-terminal of peptide monomers as Trp (for Trp-ligation) or Cys (for Thz-ligation), and a electrophile such as aldehyde at termini of the interstrand linker. Because unprotected peptides are used as starting building blocks, the need for a protecting group strategy is eliminated and thus the advantage of convenience. Unless specified, primarily Thz-ligation [59-61] is used. Other ligation methods include Trp-ligation, ψGly ligation, Michael addition ligation, disulfide linkage, or any combination thereof is also used depending on synthetic targets involved.

None of the N- or C-peptides contains Cys in their native sequences and a Cys is added to their amino terminus for chemoselective ligation. However, C34 and T1249 contain Trp at their amino terminus and Trp-ligation is used for preparing their 2α and 3α mimetics. The N-terminal Trp-rich regions of these two peptides are also postulated to be important to bind to the hydrophobic cavity of their corresponding N-helix. The Trp-ligation using Trp at the ligation site enhances binding to the hydrophobic cavity.

Poor solubility of protein mimetics frequently rises as a problem for their application as pharmaceutical compositions. Although 2α and 3α mimetics are aqueous soluble, mutation of solvent-accessible region of C-peptides are performed to further increase solubility and activity. Details of the mutation experiment are given in Example 6 infra.

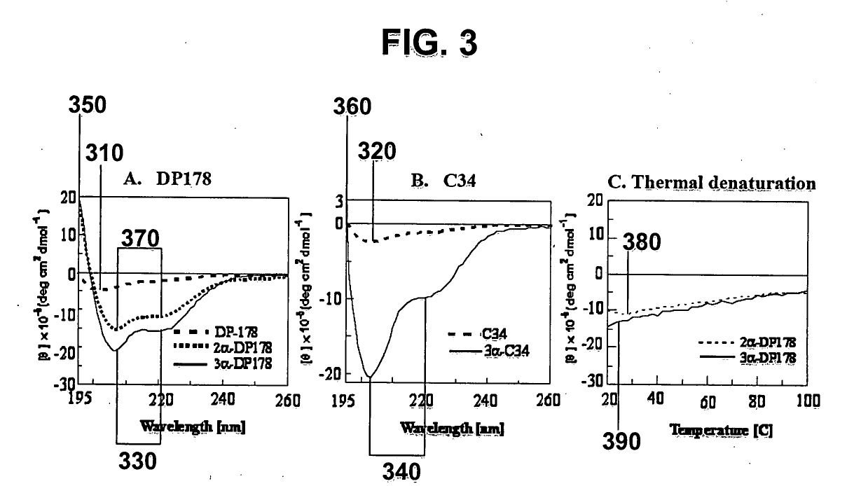

Using linker IL-1 or IL-2 in chemical ligation, 2α- and 3α-protein mimetics of monomeric peptides DP178, C34, and N-36 are synthesized. Circular dichroism (CD) measurements are used to determine structures of 3α protein mimetics. The correlation between α-helicity and CD pattern has been well established and provides reliable information for comparison. As illustrated in FIG. 3, corresponding to dashed lines 310 and 320 respectively DP178 and C34 are unstructured and displayed low helicity in aqueous solutions, characterized by a single broad negative ellipticity centered at 202 nm indicative of unordered structure [37]. In conformation promoting solvents such as TFE, they are helical (the corresponding data is not shown here). In contrast, both 3α-mimetics of DP178 and C34 are highly structured in neutral aqueous solutions, exhibiting a double minima at 208 and 222 nm corresponding to solid lines 330 and 340 respectively along with a strong positive ellipticity at 195 nm corresponding to 350 and 360 respectively, features that are typical of α-helices. 2α-DP178 behaves similarly as 3α-DP178 as demonstrated by the dotted line 370 with double minima at 208 and 222 nm. Both 2α- and 3α-DP178 are remarkably stable with no sign of denaturation at temperatures >95° C. as demonstrated by dotted line 380 and solid line 390 respectively. Table 1 summarizes the helicity based on experimental and calculated values. Both 3α-DP178 and 3α-N36 are 100% helical.

The 3α-mimetics are aqueous soluble and stable in physiological conditions containing various combinations of serum and buffered media, showing no evidence of degradation or aggregation after 24-48 hours as determined by HPLC (High Performance Liquid Chromatography). In contrast, their monomers form various orders of aggregates under similar experimental conditions. These results provide support of increased stability for 2α and 3α mimetics.

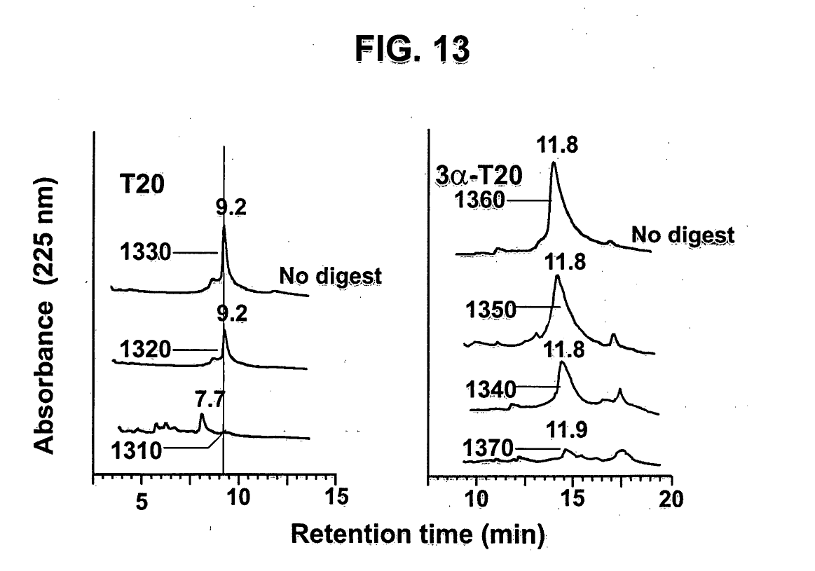

The engineered coiled-coil trimers of N- or C-peptides according to one embodiment of the present invention are substantially more resistant to both exo- and endo-proteases than their monomers. As illustrated in FIG. 13, peak intensity of certain retention time at 225 nm of each HPLC traces correlates to the amount of undigested protein or peptide. Higher peak means more protein or peptide present. For T20, peak intensity at 9.2 minutes decreased significantly from the level at no digestion 1330 to 1320 when 0.02 μg/mL of proteinase K is added and the incubation time is 1 minute. The peak diminished almost completely after 5 minutes of incubation 1310. For 3α-T20, however, when 0.2 μg/mL of proteinase K, which is ten times more concentrated than 0.02 μg/mL is added and the incubation time is 10 minutes, peak intensity at 11.8 minutes retention time 1350 only decreased slightly as compared to the peak height at no digestion 1360. A further modest decrease is observed after 60 minutes of incubation 1340. Only when the incubation continued until 1000 minutes, dramatic peak intensity decrease is observed 1370. Protein mimetics such as 3α-T20 are therefore, at least >600 fold more resistant than T20 to degradation imposed by a broad-spectrum endopeptidase proteinase K. The 3α-T20 is blocked at its N-terminal by the interstrand linker, which also prevents proteolytic degradation by amino peptidases. Together with CD spectra experiments, increase in aqueous solubility and dye-binding experiments (ANS), the results suggest that the coiled-coil mimetics allow interstrand stabilization of their hydrophobic faces thereby exposing their solvent-accessible surfaces that increases solubility and minimizes aggregation.

The antiviral activities of mimetics are determined using the single-cycle MAGI assay of a T-tropic (R8) and a M-tropic (BAL) HIV-1 on P4 cell line, a HeLa cell clone engineered to express CD-4 and integrated LTR-lac Z reporter construct [56]. Table 1 summarizes their results.

Against the R8 virus, the IC₅₀s for DP178, 2α-DP178 and 3α-DP178 are 76.4, 0.42 and 1.4 nM, respectively, which gives a 182-fold increase for the 2α mimetic and a 54-fold-increase for 3α mimetic. A similar trend is observed for the infectivity assays against the M-tropic HIV (BAL) in which the 2α- and 3α-DP178 are 14 and 20 fold more potent than that of DP 178. The IC₅₀ of C34 is 43.6 nM against the R8 virus, similar to DP178. In contrast, the IC₅₀s of 2α- and 3α-C34 are 1.02 and 1.0 nM, respectively, resulting in a 42-fold increase of potency over C34. Against the BAL virus the 2α- and 3α-C34 are even more potent, displaying IC₅₀ values of 0.09 and 0.06 nM respectively, a 45- and 88-fold increase over C34.

TABLE 1

| | | IC₅₀ (nM) | | |
|---|---|---|---|---|
| Compound | α-helicity (%) | Cell-Cell fusion | R8 virus | BAL virus |
| DP178 | 16 | 22.1 | 76.4 | 3.9 |
| 2α-DP178 | 66 | 0.18 | 0.42 | 0.28 |
| 3α-DP178 | 100 | 0.6 | 1.4 | 0.2 |
| C34 | 15 | 34.5 | 43.6 | 5.3 |
| 2α-C34 | 52 | 0.47 | 1.2 | 0.09 |
| 3α-C34 | 78 | 1.0 | 1.0 | 0.06 |
| N36 | 54 | >1000 | 910 | 105 |
| 3α-N36 | 100 | 39 | 55.4 | 7.2 |

The inhibitory effect of DP178, C34, or their mimetics on HIV-1 mediated cell-cell fusion is determined by measuring syncytium formation and the results are also summarized in Table 1. Against the R8 virus, the EC₅₀s for DP178, 2α-DP178 and 3α-DP178 are 22.1, 0.18 and 0.6 nM, respectively, which gives a 122-fold increase for the 2α mimetic and a 37-fold-increase for 3α mimetic. The EC₅₀ of C34, 2α- and 3α-C34 are 34.5, 0.47, and 1.0 nM, respectively, resulting in a 74-fold increase for the 2α mimetic and a 34-fold-increase for 3α mimetic over C34. Consistent with the literature results, as summarized in table 1, the N-peptides such as N36 are less potent than the C-peptides. Also consistent with the results obtained from C-peptide mimetics, the 3α-N36 mimetic exhibited a significant increase in potency with IC₅₀s of 7.2 and 55.4 nm.

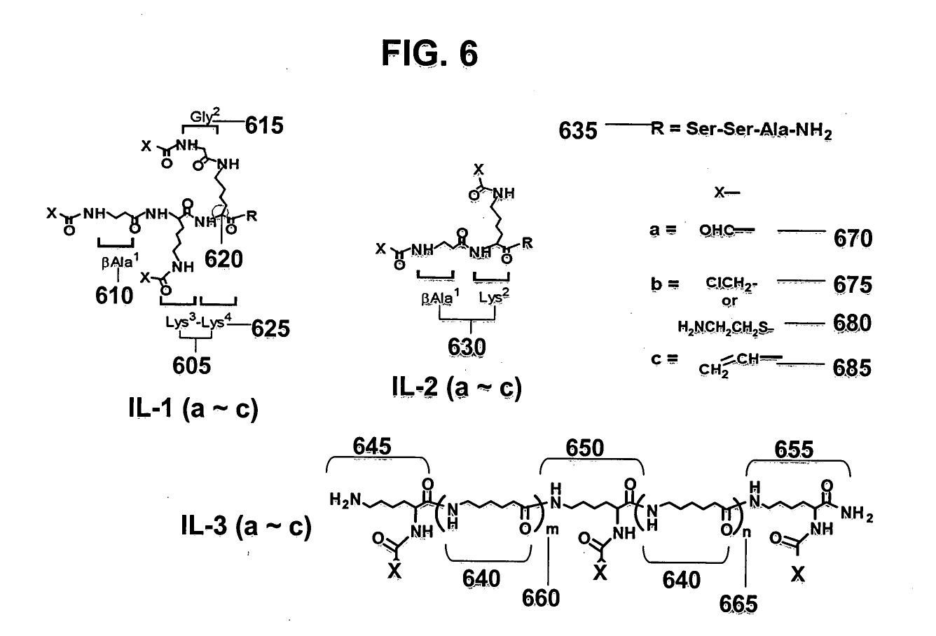

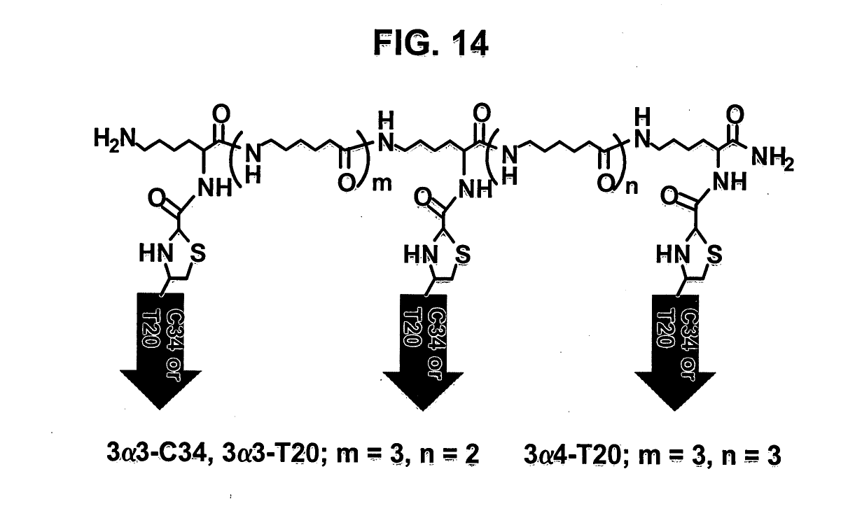

Using linker IL-3 illustrated in FIG. 6 and Thz-ligation, protein mimetics 3α3-C34, 3α3-T20, and 3α4-T20 are synthesized and structures illustrated in FIG. 14. Most recent experimental results as summarized in Table 2 indicate the mimetics are active against different isolates with IC₅₀s ranging from 0.15 to 10.18 nM.

TABLE 2

| | Tropic | IC₅₀ (nM) | | |
|---|---|---|---|---|
| Virus | Strain | 3α3-C34 | 3α3-T20 | 3α4-T20 |
| NL4-3 | X4 | 0.73 | 1.84 | 2.28 |
| 89.6 | R5X4 | 10.18 | 2.18 | 2.98 |
| pNLHxB | X4 | 0.76 | 1.10 | 2.21 |
| 92MW965.26 | R5X4 | 0.16 | 0.14 | 0.39 |
| 92UG024.2 | X4 | 0.60 | 0.52 | 1.01 |
| 92HT599.24 | X4 | 0.25 | 0.15 | 0.34 |
| T20-resistant virus | R5 | 0.47 | 1.11 | 2.17 |

More remarkably, T20 resistant viruses are sensitive to these mimetics as suggested by data listed in Table 3 in which Cys-C34 is C34 with an N-terminal Cys. The multimeric nature of the 2α- and 3α-mimetics increase avidity by binding to two different grooves of N36 helix to overcome T20-resistant viruses because mutations generally occur at the N-helix region. The monomeric T20 does not have such an advantage. These findings provide evidence for fundamental questions relating to HIV fusion events.

TABLE 3

| | IC₅₀ (nM) | | |
|---|---|---|---|
| | NL4-3 (MT-4) | T-20 Resistant Virus | Ratio |
| T20 | 4.31 | 32.32 | 7.50 |
| 3α3-T20 | 1.56 | 1.13 | 0.73 |
| 3α4-T20 | 2.46 | 1.78 | 0.72 |
| Cys-C34 | 0.71 | 4.50 | 6.34 |
| 3α3-C34 | 1.59 | 0.34 | 0.21 |

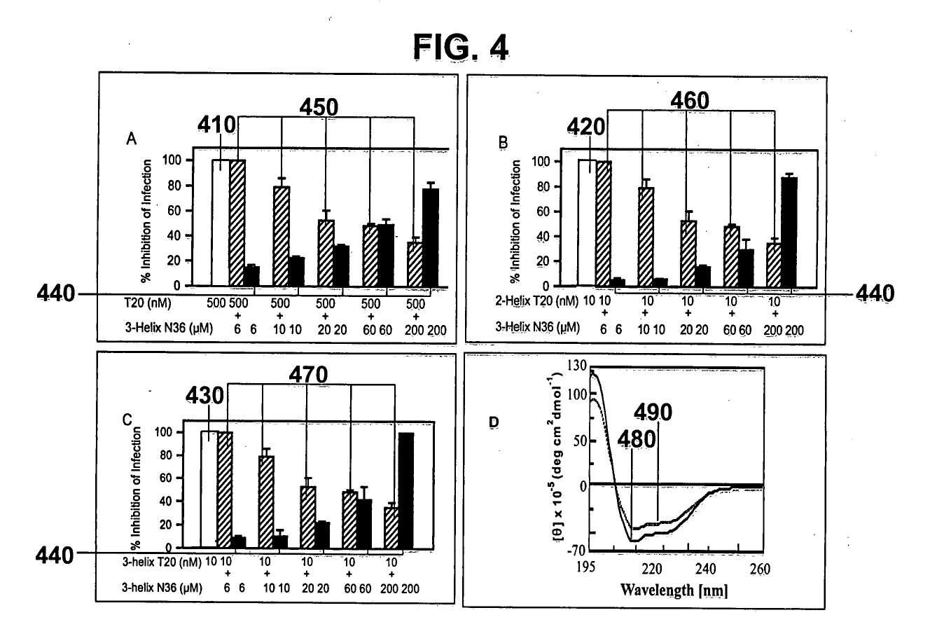

To confirm that the 2α- and 3α-C-peptides inhibited membrane fusion by binding to the N-peptides and thus preventing its transition to the 6-helix bundle, three peptide-mixing experiments were performed. First, N36 antagonized the antiviral activity of 2α- and 3α-T20. As illustrated in FIG. 4A-C, the percentage of inhibition of cell infection is close to 100% when T20 (500 nM), 2α-T20 (10 nM), or 3α-T20 (10 nM) alone is used, as represented by open bars 410, 420, and 430 respectively. Represented by solid bars 440, the percentage of inhibition by 3α-N36 increases proportionally from around 10% to close to 100% in response to increased concentration from 6 μM to 200 μM respectively. Conversely, when increased amount of 3α-N36 is added, the inhibitory effect of T20, 2α- and 3α-T20 decreased proportionally, as demonstrated by hatched bar groups 450, 460, and 470 respectively. Second, as illustrated in FIG. 4D, the helical nature of 3α-DP178 is not disturbed by the addition of 3α-N36 as indicated by comparison between the solid line 480 for 3α-DP178/N36 and dotted line 490 for 3α-DP178 only. Finally, the complexes formed by the 2α- and 3α-DP 178 with 3α-N36, as determined by biosensor analysis using a BIAcore instrument show high-affinity interactions with mean dissociate constants K_{D} of 1.2×10⁻¹¹ and 1.4×10⁻¹² M, respectively.

The antiviral and peptide mixing experiments support the contention that the 2α- and 3α-mimetics of C-peptides refold to reverse their hydrophobic faces for high-affinity binding to the N-helix of gp41. They also suggest a similar pathway in the collapse of the C-helix of gp41 to the 6-helix bundle in its fuseogenic state without the assistance of gp120. Such a process is likely sequential, proceeding by docking first at the solvent-exposed faces, followed by dissociation of the C-helix to a 2-helix bundle with a C-peptide intercalated into the central N-helix. Thus, when these mimetics intercalate into two grooves of the central N-helix, their increased avidity results in enhanced potency over the corresponding monomers.

To correlate structure-function and further define binding residues, a small series of truncated 2α and 3α mimetics based on the lead compounds DP178 and C34 of HR2 region are studied and results listed in Table 4. The amino acid sequences of these mimetics are more specifically defined in FIG. 2 as 250, 255, 260, and 265 correspond to C27, C16, C24, and C13, respectively. These peptides ranging from 13 to 27 residues are essentially inactive at concentrations 1 μM. In contrast, their 2α and 3α mimetics exhibit substantial antiviral activities. Depending on the viral isolates, several show activity at low nM concentrations. For example, 3α-C27 is active against BAL viruses with an IC₅₀ of 0.5 nM. Surprisingly, the highly shortened 2α-C13 is active against R8 virus with an IC₅₀ of 310 nM. As expected the truncated peptides are unstructured in aqueous solutions. However, they showed significant helical structures in their 2α and 3α forms, with helicity decreases with peptide lengths. These results provide further support for the findings that an increase of helical stability of gp41 peptides as protein mimetics leads to a sharp increase in potency. In summary, shortened 2α and 3α mimetics display significant potency, even when their monomers show no activity at concentrations 1 μM.

TABLE 4

| | | IC50 (nM) | | |
|---|---|---|---|---|
| Compound | α-helicity (%) | Cell-Cell fusion | R8 virus | Bal virus |
| DP178 | 16 | 22.1 | 76.4 | 3.9 |
| 3α-C24 | 34 | 290 | 310 | 36 |
| 3α-C13 | 42 | 260 | 430 | 430 |
| 2α-C24 | 55 | 38 | 80 | 67 |
| 2α-C13 | 46 | 290 | 310 | 430 |
| C34 | 15 | 34.5 | 43.6 | 5.3 |
| 3α-C27 | 9.8 | 2.8 | 380 | 0.5 |
| 3α-C16 | 9.0 | >1000 | >1000 | 124 |

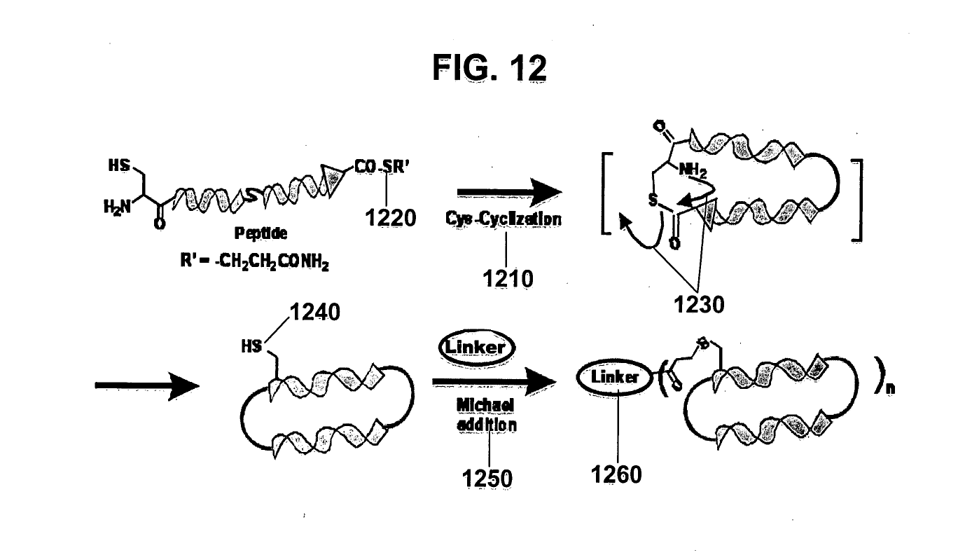

With a single constraint, a shortened monomer fray in the unconstrained end. Shortened 2α and 3α mimetics with two interstrand constraints to stabilize the putative coiled-coil structures represent a novel design because their unusual architecture of containing only parallel strands. They differ from analogous cyclic peptides that lack the tertiary structural features and cyclic proteins that contain antiparallel strands in their folded structures. The synthesis of double-constraint mimetics requires tandem ligation for their preparation to couple two interstrand linkers in tandem. Recently, the applicants' laboratory has developed several such tandem ligation schemes that are suitable for preparing such double constraint mimetics [50, 63-65]. Details of the experiment are given in Example 7 infra. Cyclic peptides as fusion inhibitors have been developed by several laboratories using rational design or phage display libraries [11]. In particular, the cyclic D-peptides developed by Kim's laboratory have the advantage of increased metabolic stability. The applicants therefore prepared cyclic peptide protein mimetics using cyclic peptides as monomeric building blocks. Cys-ligation as illustrated in FIG. 12 combined with ψGly-ligation or Michael addition is used to synthesize cyclic peptide protein mimetics.

Previous studies in designing small peptides have not afforded molecules as potent as C34. For example, Jin et al. [71] added helix-capping sequences to the 19-residue of the N-terminal portion of C34 to afford a peptide with stable helical structure that results an IC₅₀ of 1 μM in fusion inhibition. Double constraint/cyclic peptide protein mimetics represent a novel design that provides the needed stability to coiled-coil structures of shortened helical sequences of N- or C-peptides. Double constraint at both termini and cyclic peptide also has the advantage of decreasing proteolytic degradation by amino or carboxyl peptidases to increase bioavailability. More importantly, potent shortened peptides derived from different regions of gp41 provide a repertoire of fusion inhibitors to overcome HIV resistance. Low nano molar antiviral activity can be expected from the double constraint/cyclic peptide protein mimetics. Their developments facilitate further modifications to simplify their structure and represent a direction for developing small and metabolically stable antiviral fusion inhibitors. Another consideration in developing these double constraint/cyclic peptide mimetics is to determine their mechanism of actions. The mechanism of single-constrained mimetics is likely due to dissociation of their dimeric or trimeric structure to permit binding to either N- or C-helix. This mechanism may not be possible for the double constrained mimetics with short interstrand linkers (e.g. disulfide bridges) and cyclic peptide mimetics consisting of cyclic peptides as monomers.

The protein mimetics can also be used to raise monoclonal or polyclonal antibodies that bind to the coiled-coil cavity. They can further be used, either alone or in combination with other materials, in a vaccine, which will elicit the production of antibodies that bind to the coiled-coil in the individual to whom the vaccine is administered, and thereby offer protection against infection. The protein mimetic can also be used to identify from humans, other animals or antibody libraries or to raise monoclonal or polyclonal antibodies that bind to the N-helix or C-helix coiled-coil. This provides the basis for a diagnostic method in which the protein mimetic is used to assess the presence or absence of antibodies that bind the N-helix or C-helix coiled-coil in a biological sample (e.g., blood).

Several studies have used various gp41 peptides as immunogens to study the HIV entry mechanisms and for evaluation as potential vaccine candidates [75-77]. These peptides are immunogenic and their antisera are capable of immuno precipitation of gp41 and HIV virion. However, these antisera show no significant inhibition of viral infectivity in conventional assays at 37° C., but show some inhibitory activity by the suboptimal temperature method at 31.5° C. at which the virus has prolonged fusion intermediate state. In clinical trials of T20, T20 induced antibodies have also found to exert minimal effects on its efficacy [24]. Recent studies have shown the IC₅₀ of the tight-binding anti-N35CCG -N13 specific antibody fraction from gp41 HR1 region is comparable to that of the broadly neutralizing, gp120 targeted, monoclonal antibody 2G12 which has just entered phase I clinical trials. These data suggest that the trimeric coiled-coil of gp41 is accessible to neutralizing antibodies in the pre-hairpin state. Preliminary results obtained by practicing the present invention show that selected 3α-mimetics elicit antisera that recognize HIV virion, gp160 and gp120 using the sub-optimal temperature. In immuno-fluorescence analysis, they recognize HIV-1 infected P4 cells (NL-43, BAL) that displays envelope glycoprotein on their cell surfaces, confirming the specificity of 3α-mimetic induced antisera to recognize conformational Env epitopes. The quality of antibodies of the protein mimetics are superior than those obtained from synthetic peptide immunogens because of their better mimicry to the fuseogenic state of gp41 and stability as trimeric coiled-coils under various immunization methods. The monoclonal antibody 2F5 (a neutralizing antibody) and human infected pool sera strongly react with 3α-DP178 that contains a potential neutralizing epitope (ELDKWA). This 2F5 epitope is highly conserved of the monoclonal 2F5 antibody that neutralizes HIV-1 viruses across different clades and has been a target for vaccine development. However, unlike previous studies, 3α-mimetics consisting mostly truncated DP178 are poor immunogens and elicit only low-titer responses after a short 4-week immunization regimen. In addition when the protein mimetic-specific antibodies are mixed with protein mimetics in the presence of HIV-1, they show no significant neutralization of the inhibition of viral infectivity in MAGI assays at 37° C. The data obtained is summarized in table 5. A plausible explanation is antibodies with affinity for protein mimetics at micro molar range are too weak to disrupt the nano molar tight affinity binding between protein mimetics and gp41 helix.

TABLE 5

| | | Neutralization (%) | | | |
|---|---|---|---|---|---|
| Pooled Sera | Dilution | 2α-C34 | 3α-C34 | 2α-T20 | 3α-T20 |
| Pre-immune | 1,000 | <5 | <5 | <5 | <5 |
| Anti-3α-C34 | 10,000 | <5 | <5 | <5 | <5 |
| Anti-3α-C34 | 1,000 | <5 | <5 | <5 | <5 |
| Anti-3α-C34 | 100 | <5 | <5 | <5 | <5 |
| Anti-3α-T20 | 10,000 | <5 | <5 | <5 | <5 |
| Anti-3α-T20 | 1,000 | <5 | <5 | <5 | <5 |
| Anti-3α-T20 | 100 | <5 | <5 | 6 | 9 |

Despite the effectiveness of post-entry anti-retroviral drugs, there is a need for developing pre-entry drugs to counter their toxic side effects, resistance and intolerance observed in 20% of AIDS patients. Devised to exploit the fusion mechanism mediated by gp41 and to capitalize on their X-ray crystal structures, leads of potent inhibitors already in literature or clinical trials, and the applicant's strengths in chemoselective ligation, the protein mimetic approach integrates these elements in designing pre-entry protein mimetic therapeutic. The protein mimetic therapeutic can result in wider affordability and substantial clinical benefit to AIDS patients. The approach is relevant to other membrane fusion events that are mediated by trimeric coiled-coil proteins. The gp41 hairpin structure is similar to fusion proteins from several virus families. These include retroviruses, corona viruses, orthomyxoviruses and paramyxoviruses. Some of these viruses such as Ebola and influenza are attracting attention because of the threat of bioterrorism and newly emerging infectious diseases such as severe acute respiratory syndrome (SARS). In addition, gp41 also shares similarity with v-SNARE and t-SNARE, protein complexes involved in vesicle fusion [51-53], suggesting that hairpin formation may be common to a wide range of membrane fusion events. In short, protein mimetics can be used as entry inhibitors to treat human diseases.

### METHODS, EXAMPLES AND IMPLEMENTATIONS

Without intent to limit the scope of the invention, exemplary methods and their related results according to the embodiments of the present invention are given below. Note again that titles or subtitles may be used in the examples for convenience of a reader, which in no way should limit the scope of the invention. Moreover, certain theories are proposed and disclosed herein; however, in no way they, whether they are right or wrong, should limit the scope of the invention.

### EXAMPLES

### Example 1

### Design and Synthesis of Monomeric Peptides

Unprotected peptides are used as the starting materials. Their amino acid sequences are derived from either the HR1 (N-peptide) or HR2 (C-peptide) region of gp41 as illustrated in FIG. 2. For N-terminal specific ligation, unprotected peptide monomers must have at their N-terminus a specific amino acid residue. In this case, Trp that occurs naturally in the target sequences or Cys that is placed intentionally in their sequences is included. These peptides are then used for two N-terminal specific ligation methods, Cys for Thz-ligation and Trp for Trp-ligation. The unprotected peptide monomers are prepared by solid-phase synthesis. All peptides are synthesized by a stepwise solid-phase method using either tert-butoxycarbonyl (Boc) or fluorenylmethoxy carbonyl (Fmoc) chemistry [57, 58]. All peptides are purified by HPLC and characterized by mass spectrometry.

### Example 2

### Design and Synthesis of Interstrand Linkers

Based on convenience and flexibility, interstrand linkers use branching lysine as primary building blocks. Other interstrand linkers may also be utilized to practice the present invention. As illustrated in FIG. 6 interstrand linker IL-1 designed for 3α mimetics contains a di-Lys 605 with additions of β-Ala 610 and Gly 615 to make each of the three arms pseudo symmetrical: each peptide chain is tethered 10 atoms from the α-carbon 620 of Lys⁴ 625. Similarly, the interstrand linker IL-2 designed for 2α mimetics is based on β-Ala¹-Lys² 630. The flexibility of an interstrand linker with many rotatable CH₂ bonds is useful to accommodate turns for interlocking two or more coiled-coils. The Lys-based design of IL-1 and IL-2 is devoid of side chains that reduce steric hindrance when binding to the N- or C-helix and minimizes unwanted immune responses as immunogens. To increase aqueous solubility, a tri-peptide Ser-Ser-Ala 635 is added to the carboxylic terminus. Other peptides such as part of N- or C-peptides or surface modifiers are used to fine tune the design of the interstrand linkers. Among them, a linker made out of 5-amino-hexanoic acids 640 linked lysines, Lys¹ 645, Lys² 650, and Lys³ 655 demonstrates superior properties, where “m” 660 and “n” 665 can be any integrals equal to or greater than one. Various functional groups can be introduced to the amino termini, including aldehyde 670, chloroacetyl 675, β-aminoethyl thiol 680, and acrylate 685.

In Thz-ligation, the interstrand linkers contain a Ser at amino terminus that is converted to an aldehyde by periodate oxidation at pH 5.5. In one embodiment, the following procedure is followed. The N-terminal Ser is oxidized by adding 8 mol equivalent of sodium periodate to the scaffold precursor in pH 5.5, 0.2 M acetate buffer (100 μL/mg). After 10 min, the reaction mixture is purified on HPLC to remove formaldehyde formed in the reaction and excess oxidant. The purified aldehyde-scaffold is obtained in >90% yield. These aldehyde functionalized interstrand linkers are then used for ligation with the appropriate peptide monomers to form protein mimetics.

### Example 3

### Thz- and Trp-ligation

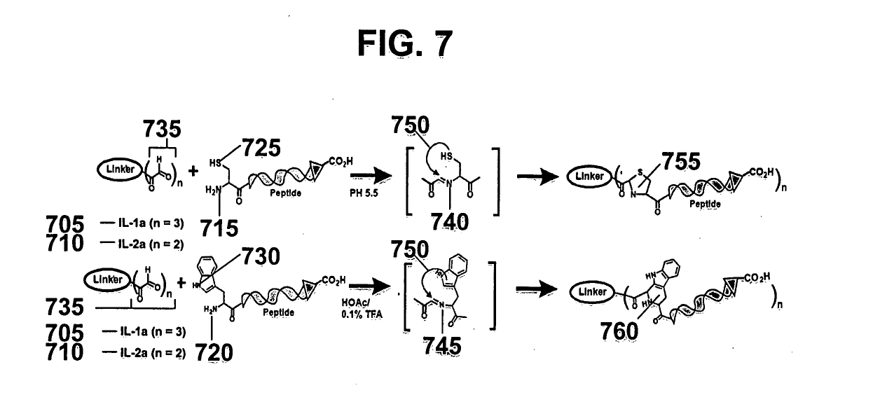

As illustrated in FIG. 7, Thz- and Trp-ligation require aldehyde of IL-1a (705) or IL-2a (710) to ligate to a Cys- or Trp-containing peptide. A α-amino group such as 715 or 720 and a side chain functional group of an N-terminal Cys 725 or Trp 730 are absolutely required to form a heterocycle. The ligation starts with the aldehyde group 735 from the linker reacting with the α-amino group to form an imine 740 or 745, which isomerizes 750 to a five-membered 755 or a six-membered 760 heterocycle. Thz-ligation is very facile and is performed in aqueous solutions in pH range 1-8 and Trp-ligation is best performed under acidic conditions with pH<3 using glacial HOAc and a catalytic amount of trifluoroacetic acid.

In a typical Thz-ligation to form a 2-helix-bundle or a 3-helix-bundle, 2.0 or 3.3 molar excess of Cys-peptide respectively is mixed with aldehyde-scaffold in a 10% acetonitrile/water buffer containing 0.1% of TFA at pH 2. The reaction mixture is deaerated and kept under nitrogen. The reaction process is checked by HPLC and confirmed by MS. General reaction finishes in 24 hours with the 2-helix-bundle or 3-helix-bundle as major product. The bundles are then purified by HPLC in the yield of 40-60%. Concentrations of protein mimetics are measured by tyrosine and tryptophan UV absorbance at 285 nm.

The Cys- and Trp-ligation are semi orthogonal and can be used in tandem to prepare chimeric protein mimetics. The specificity of these ligations has been studied with a di-peptide library containing 400 di-peptides in the applicants' laboratory. N-terminal Cys reacts immediately and is about 10,000 times faster than Trp, which requires 100% acetic acid as solvent. Thus, Trp and Thz ligation can be achieved consecutively for tandem ligation to form chimeric 2α and 3α mimetics consisting of both N- and C-peptides in a single molecule, first by Thz ligation in aqueous solvents and then by Trp ligation in glacial HOAc. Interestingly, chimeric 2α or 3α mimetics have been shown to be potent inhibitors and are postulated to be a new class of fusion inhibitors that may have a different binding site than their homomeric mimetics [36].

### Example 4

### ψGly Ligation

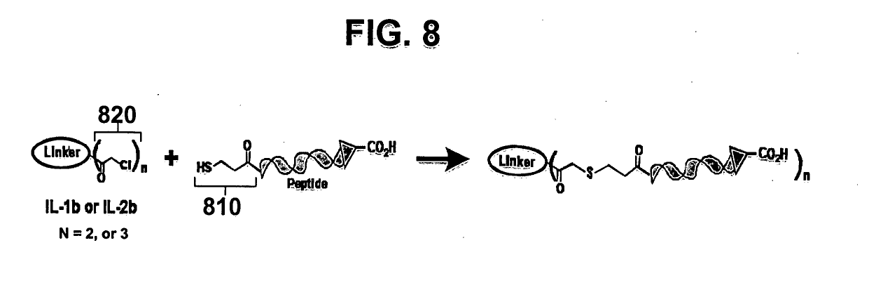

A disadvantage of the Thz- or Trp-ligation mediated through imine chemistry is the generation of a stereogenic center at the ligation site as a pair of diastereomers. For 3α mimetics containing three ligation sites, a mixture of 16 diastereomers is obtained. These mixtures are not anticipated to affect biological and immunological studies, but they become problematic in biochemical studies such as NMR and X-ray crystallography that requires optically pure compounds. To eliminate the disadvantage associated with imine-mediated ligation, ψGly ligation is used. As illustrated in FIG. 8, nucleophile 3-mecapto propionate 810 and electrophile chloroacetyl 820 undergo thioalkylation to afford a ψGly at the ligation site. The nucleophile and electrophile functional groups can be interchanged in this scheme. ψGly ligation of protein mimetics is not as efficient as Thz- or Trp-ligation due to the hydrolysis of chloroacetyl moiety that results in low yield. However, ψGly ligation is used to produce 2α and 3α mimetics of C34, DPI78 and N35 for NMR and X-ray crystallographic analyses. An interesting outcome in changing ligation chemistry is the attendant change of ligation sites. Thz- and Trp-ligation produce a proline-like mimetic at the ligation site that is more rigid than the ψGly linkage formed by the ψGly ligation. However, proline-like linkages facilitate tun-like structures of these interstrand linkers to stabilize protein mimetics. These differences in linkages provide useful comparisons for refining the design of protein mimetics.

### Example 5

### Carboxyl Terminal Ligation

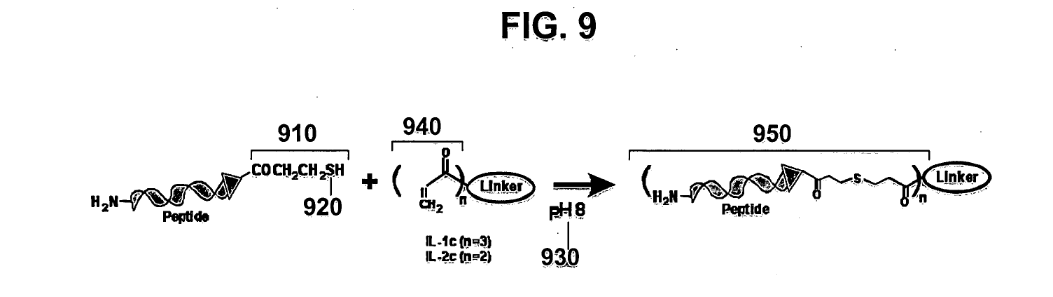

For ligation at the C-terminus, as illustrated in FIG. 9, a specific functional thiol group such as 3-mercapto propionate 910 is placed at C-terminus to afford unprotected peptides with a thiol moiety 920 at the carboxyl terminus. This thiol is then used for Michael addition at pH 8 (930) to acrylated linkers 940 IL-1c and IL-2c to form the C-terminal linked mimetics 950. Because of the six-helix hairpin structure, the polarity of interstrand linkage is important. N-linking C-peptides are more effective than the C-linked mimetics. Conversely, C-linking N-peptides are more effective than the N-linked mimetics. An alternative plan for Michael addition is to adapt ψGly ligation as described in Example 4.

### Example 6

### Mutation of Solvent Accessible Region of C-Peptide

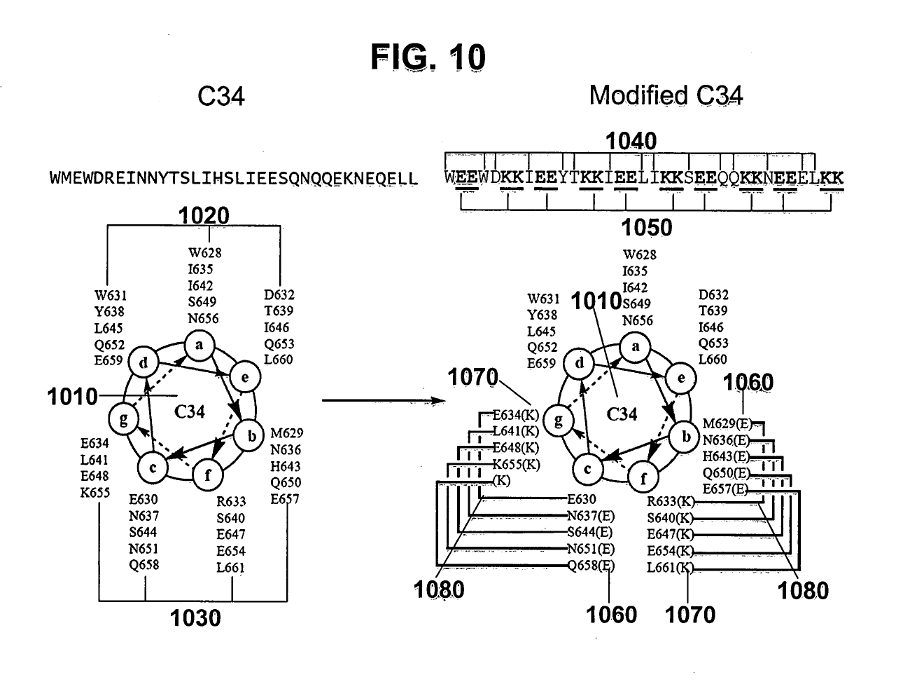

As illustrated in FIG. 10, based on a helical-wheel diagram 1010 the helical structure of the C-peptide contains two faces. A hydrophobic face consisting of hydrophobic residues 1020 may interact with the N-helical peptide while a hydrophilic region rich in glutamic acid 1030 is exposed to solvent. To improve the solubility and stability hence the activity of the C-peptides the solvent exposed surface of C-peptides is modified. Based on the helical-wheel diagram depicting the interaction of the inner coiled-coil formed by N36 and C34, C34 peptide is modeled using the following two criteria: 1. retain conserved amino acid residues critical for interaction with the inner strand formed by N36 (a, d, and e, positions 1040); 2. replace nonconserved residues locating at solvent-accessible face (b, c, f and g positions 1050) by Glu or Lys to form intrahelical salt bridge for i and i+4 positions. Consequently, ten Glu (E, 1060)-Lys (K, 1070) intrahelical salt bridges are possible on the C34 peptide 1080. Glu or Lys intrahelical salt bridges enhance solubility as well as helicity. This design is recently adopted by Otaka et al. [62], who found that such modification increased the IC₅₀ of an analog of C34 by three fold comparing to the parent compound.

### Example 7

### Design and Synthesis of Double Constraint Mimetics

Two series of truncated analogs based on DP178 (T20) and C34 are the focus of the study. Since both series are part of C-peptides (HR2) and share a 24 amino acid residue overlap (aa 638 Tyr to 661 Leu), they essentially covered the entire HR2 region. Since the C-peptides have an isoleucine/leucine zipper motif with 4,3-repeats of hydrophobic amino acids occupying the a and d positions, the truncation strategy mimic their structural features by deleting 3 or 4 residues in each analog. An important motif of these peptides is the Trp-rich region, which may form residue responsible for the binding pocket of the coiled-coil surface [66, 67]. C34 has a Trp-rich region (WMEW) at the N-terminus, whereas DP178 has a Trp-rich region at the C-terminus (WN F). Thus, truncation of DP178 is at its amino terminus to preserve its truncated analogs containing C-terminal Trp-rich region. In contrast, truncation is at the carboxyl terminus of C34 to preserve the N-terminal Trp-rich region.

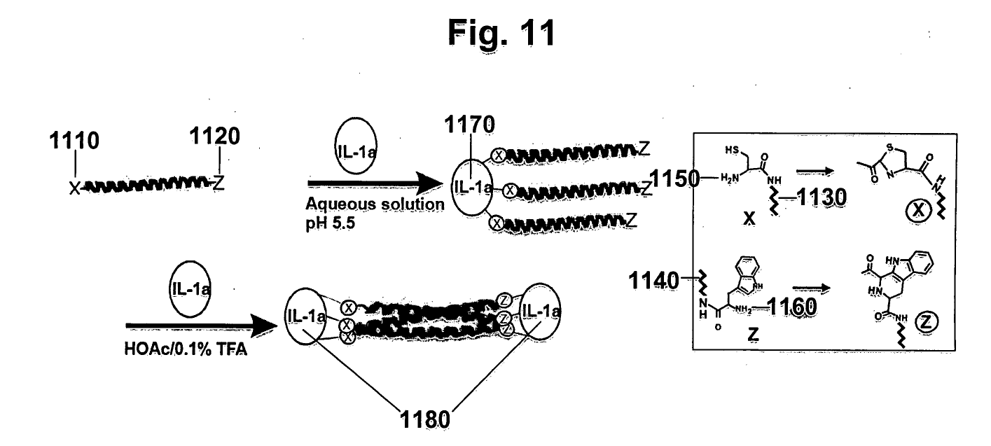

The combination of Thz- and Trp-ligation to constrain in tandem both ends of the N-terminal Trp-containing monomers are used to prepare the double constraint protein mimetics. As illustrated in FIG. 11, the monomer contains a Cys at carboxyl terminus 1110, linked to the amino side chain of a Lys at the end of the monomeric peptide and Trp 1120 at the amino terminus. The carboxyl groups of the Cys 1130 and Trp 1140 are linked to the peptide monomer, leaving the amino groups on Cys 1150 and Trp 1160 respectively free to ligation. Thz-ligation of IL-1a with the monomer in aqueous conditions buffered at about pH 5 affords constraint at the carboxyl terminus 1170, followed by Trp-ligation at pH 2 again with IL-1a to afford the double constraint 1180 at both termini. Details of Thz-and Trp-ligation are illustrated in FIG. 7. This strategy is also applicable to the 2α double constraint mimetics.

Because Thz- and Trp-ligation are semi-orthogonal and unexpected difficulty due to a prolonged reaction time of >24 hours may lead to undesirable side reactions, two alternative strategies are considered. A complete orthogonal strategy is to use the tandem ligation of Michael addition for the C-terminal constraint and Trp-ligation for the N-terminal constraint. Details about Michael addition and Thz-ligation are illustrated in FIG. 9 and FIG. 7 respectively. A second alternative strategy is to use disulfide linkages as a constraint at either the amino or carboxyl end. This strategy of placing a CysCys di-peptide as an interstrand on a monomer has been exploited successfully by Carole Bewley to form trimeric coils of N-peptides [36]. Such a design places an N-terminal Cys for the 2a mimetics and CysCys di-peptide for 3α mimetics. Additionally, placing a Cys at the carboxyl terminus does not interfere with the Thz- or Trp-ligation because such a Cys lacking a free c-amine cannot form thiazolidine.

### Example 8

### Design and Synthesis of Cyclic Peptide Protein Mimetics

Cyclic peptides developed by other laboratories are constrained side chain to side chain by a disulfide bridge. For the purpose of increasing metabolic stability of cyclic peptide protein mimetics, these disulfide-constrained leads as end-to-end cyclic peptides in which their N-and C-termini are joined as a peptide bond is modified. As illustrated in FIG. 12, a facile synthesis of such cyclic peptides through Cys-ligation 1210 of their unprotected peptide thioesters 1220 is used instead [68-70]. The liberated 1230 free thiol 1240 after Cys ligation is then exploited for ψGly ligation or Michael addition 1250 to the interstrand linker 1260. Same rules used in Example 7 are followed to design monomeric peptides used in this example.

### Example 9

### Mimetics as Immunogens

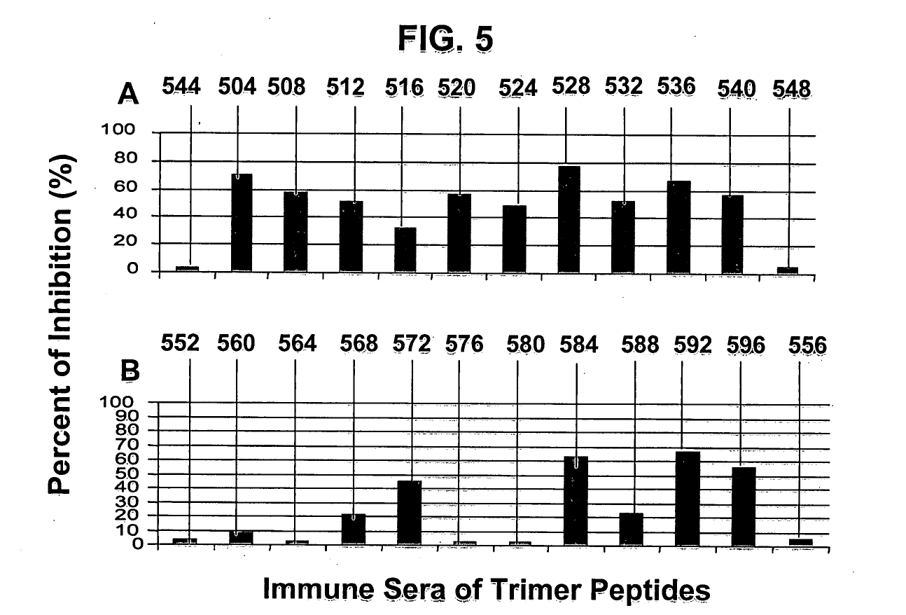

To determine whether the 3a mimetics could generate neutralizing antibodies that mimic the binding properties of these mimetics, antisera is raised from guinea pigs. After 4 weeks of immunization, these antisera from 3α-N36 (sera S5 and S7), its 11-residue truncated version (sera S22 and S23) as well as three truncated forms of 3α-C34 (S18, S20 and S26) are able to immuno precipitate virus particles, gp160 and gp41 as well as the protein mimetics. Furthermore, several of these sera are able to inhibit infectivity of R8 and BAL viruses as determined by the MAGI assay. As illustrated in FIG. 5, HeLa cell line (MAGI) expressed CD-4, CXCR4 and CCR5 receptors are used to measure biological activity of anti-trimer peptides immune sera in HIV-1 infection. MAGI-CCR5 cells containing the HIV-LTR-gal, are treated with pre-incubating immune serum with HIV-1 R5 and X4 viruses, respectively, first incubate at 31.5° C. for 4 hours, then at 37° C. Neutralization of gp41 trimer peptide immune sera for T-tropic HIV-1 (R8) is shown in panel A. 30-78% inhibition is observed from treated sera, S5 (504), S7 (508), S18 (512), S20 (516), S22 (520), S23 (524), S26 (528), S5+S18 (532), S23+S26 (536), S5+S20 (540), where medium (544) and pre-immune serum (548) serve as controls that show minimal inhibition. Neutralization of gp41 trimer peptide immune sera for M-tropic HIV-1 (HIV-1BAL) is shown in panel B. Minimal inhibition similar to controls medium 552 and pre-immune serum 556 are observed in S7 (564), S22 (576), and S23 (580). 10-20% of inhibition is observed in S5 (560) and S18 (568). The combination of the S5 and S18 doesn't improve the percentage of inhibition S5+S8 (588). 45-68% of inhibition is observed in S20 (572), S26 (584), S23+S26 (592), S5+S20 (596). Data are expressed as the percent of blue cells number per culture well compared to the level in a parallel control in which virus is pre-incubated with no serum.

### Example 10

### General Characterization of Mimetics

All mimetics are characterized by the following assays to determine unless specified otherwise, among other features, their inhibitory potency, toxicity, solubility and stability to exo-peptidases. The single-cycle MAGI assay is used to determine their inhibitory potency on HIV-1 infectivity using a T-tropic (R8) and a M-Tropic (BAL) HIV-1 on P4 cell line, a HeLa cell clone engineered to express CD4 and integrated LTR-lac Z reporter construct [56]. Their inhibitory potency of the HIV-1 mediated cell-cell fusion is determined by syncytia formation. The toxicity of mimetics is determined by hemolytic assay on fresh human erythrocytes (membranolysis). The peptide concentrations causing 50% hemolysis (EC₅₀) are calculated from the resulting dose response curves. If they are found to be hemolytic, their cyto toxicities are determine on HeLa by MTT or trypan blue stain method in conjunction with glucose-exclusion assays. Their membranolytic or membrane fusion actions induced by the mimetics, liposome aggregation and vesicle leakage are employed for observing their effect on liposomal membranes prepared by the extrusion method. Promising mimetics with inhibitory potency in low nanomolar or subnanomolar concentrations are further evaluated by the following examples.

### Example 11

### Activity Against HIV-1 Mediated Membrane Fusion in Diverse Isolates and Cell Types

Previous study reveals that there is a considerable variability in the sensitivity of primary isolates to T20 [24]. The concentration of T20 needed to inhibit primary virus isolates can vary by two logs. Furthermore, susceptibility of T20 is also influenced by coreceptor usage [31]. For example, T20 sensitivity can be modulated by CCR5 coreceptor expression level and is more potent to target cells with lower levels of CCR5. These studies are duplicated on protein mimetics to compare with T20. Studies of entry inhibitors typically have utilized cultured cell lines or peripheral blood mononuclear cells (PMBC). However, HIV-1 replicates in additional cell types such as dendritic cells (DC) and cord blood mononuclear cells (CBMC) that have important implications for therapy. 2α and 3α mimetics are examined on PBMC, CBMC, macrophages, and mature and immature DC. The inhibition is performed according to published methods [72] using subtype B R5 primary isolates HIV-1 for 5 to 7 days and the extent of viral replication is determined by p24 antigen enzyme-linked immuno sorbent assay of the culture supernatants. For comparison, T20 and Rantes are used as controls. Results obtained are listed in Tables 1 and 4, respectively.

For compounds made with IL-3 as illustrated in FIG. 6 and FIG. 14, P4R5 cell line, a HeLa cell clone engineered to express CD4 and CCR5 and an integrated LTR-lacZ reporter construct is used to detect HIV-1 infection. HIV-1 stocks are diluted in D10 medium, and then increasing concentrations of compounds are added. X-Gal staining is used to detect infected cells. Infected cells are quantified by counting stained cells using NIH Image software analysis of images captured with a charge-coupled device camera equipped with a macro lens. P4R5 cells are cultured in Dulbecco's modified Eagle medium. The wild-type HIV-1 molecular clones pNL4-3(X4), chimera 89.6(X4R5), pNLHxB(X4), 92MW965.26(R5), 92UG024.2(X4), 92HT599.24(X4) are used for these studies and results reported in Table 2.

### Example 12

### Viral Resistance Measurements

The optimization of a treatment strategy benefits from the knowledge of the baseline susceptibility and acquired resistance to entry inhibitors such as those mimetics proposed in this application. Previous studies have shown that viruses have developed resistance to T20 both in vitro and in vivo. Primary sites of such mutants carry substitutions in the GIV tri-peptide (aa 36-38) and in other positions of the N-helix of gp41 [29, 74]. Selected protein mimetic-resistant viruses are established for characterization of different mimetics against wild-type to the acquired drug-resistant viruses. The selection of viruses resistant to 2α- and 3α-DP178 and C34 are derived by repeated passage of the uncloned HIV-1_{IIIB} through the CEM-4 cell line in the increasing concentrations of the mimetics. DP 178 is used as a control. The concentrations of mimetics that reduce the wild type HIV-1_{IIIB} infectious titer by >95% are used. Fresh doses of mimetics are added to the medium every 48 hours and viral production is monitored by RT activity or P24 ELISA. After 5 to 15 passages, the viruses are harvested to determine their resistance to various protein mimetics and DP178.

For compounds used in Table 3, T20-Resistant and pNL4-3 viruses are generated from MT-4 cells. HeLa-CD4/LTR-lacZ-CCR5 (P4R5) cells are used as target cells in single-cycle MAGI infection assays as previously described in Example 11.

### Example 13

### CD Study for Structure Determination

Among other things, CD studies are used for two purposes. CD provides information on the overall secondary structure of helical peptides [76-80]. CD can also be used to study the mechanism of actions of the heteromeric complexes of N- and C-helices. In aqueous solutions, the CD spectra of N36, DPI78 and C34 are mostly unstructured, but their 2α and 3α mimetics exhibit spectra typical of helical structures as illustrated in FIG. 3. CD data are used to compare monomers and their 2α and 3α mimetics to provide support for that protein mimetics provide interfacial stabilization of amphipathic peptides, leading to an increase in helicity. Their increase as measures in fractional helicities in the mimetics are calculated by the methods of Wu et al. [81] using −2000 and −32,000 deg cm²/dmol for 0% and 100% helix content, respectively. For those mimetics with low helicity content, their CD spectra are measured in 30% TFE to determine their propensity to form helix in a hydrophobic environment. The stability of mimetics exhibiting high helical content is determined by thermal denaturation monitored by CD at 222 mn.

### Example 14

### Correlation of Helical Structure with Activity

Previous studies using engineered proteins and rationally designed peptides have provided support that there is a correlation of helicity in gp41 peptides with inhibitory potency [36, 71]. Furthermore, a specific face of the helix must be exposed to block viral infectivity. A series of C-peptides with lengths ranging from 10 to 39 residues and in various forms of mono-, di-, and trimers are synthesized and tested. Furthermore, a small series of C-peptides with mutation on the solvent-accessible surfaces are also synthesized and tested. This rich repertoire of gp41 peptides affirms the correlation of helicity with inhibitory potency. All CD spectra of various mimetics are determined on a Jasco J-810 spectro polarimeter over the wavelength range of 250-190 nm using a 1.0 mm path length cell, a bandwidth of 1.0 nm, a response time of 2 sec, and averaging over three scans. Light scattering and background absorption from aggregates are eliminated by baseline subtraction. For each sample, the minimum, zero crossing and maximum regions are examined to characterize the helix components and a double minima around 209 and 222 nm and positive maximum around 192-197 nm for α-helical structures. The presence of 3₁₀-helix is determined because a synthetic peptide representing the C-terminal region of DP 178 is reported to exist as 3₁₀-helix, which is characterized by a weak negative shoulder between 220 and 230 nm and a minimum at 205 nm [82].

### Example 15

### Stability and Proteolytic Resistance of Protein Mimetics

For experiment carried out to obtain data in FIG. 13, the following procedure is followed. Proteinase K at two concentrations 0.02 μg/mL and 0.2 μg/mL in PBS are added to T20 and 3α-T20 (10 μM) in PBS respectively, and incubated at 37° C. Because the fast degradation of T20, the digestion of T20 is performed at 0.02 μg/mL of proteinase K, a 10-fold lower concentration than those (0.2 μg/mL) performed for 3α-T20. The amounts of proteinase K and incubation time are: 1 min and 5 minutes for T20, and 10, 60 and 1000 minutes for 3α-T20. The digestion was quenched by the HPLC starting buffer containing 0.05% TFA and monitored by HPLC.

### Example 16

### Characterization of Heteromeric Complex of 2α or 3α Mimetics with Their Complementary N- and C-Peptides

A key postulate of the pre-hairpin model that the N- or C-peptides act as dominant negative inhibitors is that they form heteromeric complexes to prevent transition to the six-helix bundle critical for membrane fusion. However, the binding mode of the protein mimetics to form such heteromeric complexes is different from conventional synthetic peptides and needs to be determined. Three methods are used to characterize the heteromeric complex of N- or C-mimetics. They include binding analysis by CD, quantitative analysis by biosensor, ultracentrifugation and mass spectrometry as well as neutralization infectivity assay.

The interaction between peptides can be assessed using CD, by comparing the experimental spectrum of the mixed peptides with the average theoretical non-interacting spectrum of each peptide alone as described by Lawless et al [37]. Mixing C34 with N36 is known to increase the α-helical content, indicating binding of these two amphipathic leucine/isoleucine zipper-like segments that form a complex with an α-helical structure [37, 83-84]. Similarly, mixing DP178 with N36 also resulted in attenuated CD signal, suggesting a complex of N36 and DP178. However, no perturbation of CD spectra is observed when DP 178 is added to the N34/C34 complex, suggesting that the N36/C34 complex forms a stable structure as proposed in the pre-hairpin model [83, 84]. The interaction between monomer of C34 or N36 or their truncated analogs proposed in this application with their complementary 2α or 3α mimetics to form a complex similar to N36/C34 is expected to follow literature precedents. A higher α-helix content in the experimental CD spectra than the calculated spectra suggests stable interactions of the monomers and protein mimetics. The CD spectra suggesting a complex of 2α or 3α mimetics of C-peptides with the 3α-N36 or the chimeric 4α- and 5α-N36 is complex because the lack of literature precedent. However, an increase of a-helix content and the extent of increase provide clues to their binding mechanism.

The binding parameters of the heteromeric N/C-complexes are determined quantitatively by biosensor analysis using a BIAcore instrument. The dissociate constants K_{D} are obtained from their sensorgrams. Thus far, there appears to be a strong correlation of K_{D} and inhibitory potency. Jin et al. [71] found that binding affinity was proportional to viral inhibition since their most potent C-peptide analog with an IC₅₀ of 1 μM also exhibited the highest affinity with a K_{D} of 2.9×10⁻⁷ M. For more potent 2α and 3α mimetics, preliminary results showing higher K_{D}s of <1×10⁻⁸ M in 2α-DP178 and 3α-DP178 are in line with their findings. The biosensor analysis to correlate binding and activity is particularly suited for analyzing shortened protein mimetics series because of the 3α-N36 and its chimeras as excellent mimetics of the N-helix. The biosensor analysis is also used to determine the binding mechanism of the 2α and 3α mimetics of the C-peptides to various homomeric and chimeric N36 constructs. Analytical ultracentrifugation of the heteromeric complex as monodisperse species provide the stoichiometric ratio of the N- and C-peptide with the protein mimetics. TOF-MS is also used to detect the N- and C-peptide complex. Since there is a correlation of the stability of the heteromeric complex with peak detection, this method provides clues to the binding mode of the mimetics vs. synthetic peptides. Furthermore, mass spectroscopic analysis is convenient, sensitive, and complementary to existing analytical methods.

Since the C-peptides are substantially more potent than the N-peptides that can act as antagonists, the heteromerization is determined using a neutralization assay by peptide mixing experiments of the N- and C-peptides as well as their mimetics. As shown in preliminary results illustrated in FIG. 4, N-peptides display a dose-dependent antagonistic effect on the 2α and 3α mimetics of DP178 as determined by the single-cycle infectivity assay. The most useful outcome is the determination of the binding mechanism of 2α and 3α mimetics of the C-peptides to the N-helix.

### Example 17

### NMR (Nuclear Magnetic Resonance) Studies

High-field NMR is used to analyze the complex of 2α-DP178 and 3α-DP178 with the corresponding 3α-N36 to determine their mode of binding. NMR studies are performed on 2α-C34, 3α-C34, 2α-DP178 and 3α-DP178. Their spectra compare with their monomers whose NMR spectra have been reported. The NMR spectra are collected on Bruker spectrometers operating at 600 MHz with an inverse, broadband probe. Two-dimensional spectra (DQFCOSY, TOCSY, ROESY and NOESY) are recorded under standard pulse sequences with the number of acquisition set to 64 for NOESY [85], ROESY [86] and DQF-COSY [87] and 32 for TOCSY spectra [88]. All NMR data are transferred to a workstation and processed with suitable software. Molecular modeling is performed on a Silicon Graphics Workstation running CHARMM 22 as known to people skilled in the art. Models are built by using MacKerell protein potential parameter sets. The NMR studies provide further valuable structural information of theses molecules.

While there has been shown several and alternate embodiments of the present invention, it is to be understood that certain changes can be made as would be known to one skilled in the art without departing from the underlying scope of the invention as is discussed and set forth in the specification given above and in the claims given below. Furthermore, the embodiments described above are only intended to illustrate the principles of the present invention and are not intended to limit the scope of the invention to the disclosed elements. Additionally, the references listed herein are incorporated into the application for providing background information only.

### LIST OF REFERENCES

[1]. Wyatt, R. Sodroski, J. The HIV-1 envelope glycoproteins: gusogens, antigens, and immunogens. Science 280:1884-88. 1998.
[2]. Berger E A, Murphy P M, Farber J M. Chemokine receptors as HIV-1 coreceprors: roles in viral entry, tropism, and disease. Annu. Rev. Immunol. 17:657-700. 1999.
[3]. Littman D R. Chemokine receptors: keys to AIDS pathogenesis? Cell 93:677-80. 1998.
[4]. LaBranch, C C. Galasso, G. Moore, J P. Bolognesi, D P. Hirsch, S M. Hammer, S M. HIV fusion and its inhibition. Antiviral Research 50:95-115. 2001.
[5]. Eckert, D M. Kim, S P. Mechanism of viral membrane fusion and its inhibition. Annu. Rev. Biochem. 70:777-810. 2001.
[6]. Chan, D C. Kim, P S. HIV entry and its inhibition. Cell 93:681-684. 1998.
[7]. Moore, J P. Stevenson, M. New targets for inhibitors of HIV-1 replication. Nat. Rev. Mol. Cell. Biol. 1:40-49. 2000.
[8]. D'Souza, M P. Cairns, J S. Plaeger, S F. Current evidence and future directions for targeting HIV entry: therapeutic and prophylactic strategies. JAMA 284:215-222. 2000.
[9]. Cammack, N. The potential for HIV fusion inhibition. Current Opinion in Infectious Diseases 14:13-6. 2001.
[10]. Clercq, E D. New developments in anti-HIV chemotherapy. Bioch. Biophy. Acta. 1587:258-275. 2000.
[11]. Sia, S K. Carr, P A. Cochran, A G. Malashkevich, V M. Kim, P S. Short constrained peptides that inhibit HIV-1 entry. Proc. Natl. Acad. Sci. USA. 99:14664-14669. 2002.
[12]. Lynch, C L. Hale, J J. Budhu, R J. Gentry, A L. Mills, S G. Chapman, K T. MacCoss, M. Malkowitz, L. Springer, M S. Gould, S L. DeMartino, J A. Siciliano, S J. Cascieri, M A. Carella, A. Carver, G. Holmes, K. Schleif, W A. Danzeisen, R. Hazuda, D. Kessler, J. Lineberger, J. Miller, M. Emini, E A. 1,3,4-Trisubstituted pyrrolidine CCR5 receptor antagonists. Part 4: synthesis of N-1 acidic functionality affording analogues with enhanced antiviral activity against HIV. Bioorganic & Medicinal Chemistry Letters. 12:3001-3004. 2002.
[13]. Gao, P. Zhou, X Y. Yashiro-Ohtani, Y. Yang, Y F. Sugimoto, N. Ono S. Nakanishi T. Obika S. Imanishi T. Egawa T. Nagasawa T. Fujiwara H. Hamaoka T. The unique target specificity of a nonpeptide chemokine receptor antagonist: selective blockade of two Th1 chemokine receptors CCR5 and CXCR3. Journal of Leukocyte Biology 73:273-80. 2003.
[14]. Ryu, J R. Jin, B S. Suh, M J. Yoo, Y S. Yoon, S H. Woo, E R. Yu, Y G. Two interaction modes of the gp41-derived peptides with gp41 and their correlation with antimembranefusion activity. Bioch. Biophy. Res. Comm. 265:625-629. 1999.
[15]. Root, M J. Kay, M S. Kim. P S. Protein design of an HIV-1 entry inhibitor. Science 291:884-888. 2001.
[16]. Arakaki, R. Tamamura, H. Premanathan, M. Kanbara, K. Ramanan, S. Mochizuki, K. Baba, M. Fujii, N. Nakashima, H. T134, a Small-molecule CXCR4 inhibitor, has no cross-drug resistance with AMD3100, a CXCR4 antagonist with a different structure. J. Virol. 73: 1719-1723. 1999.
[17]. Xu, Y. Tamamura, H. Arakaki, R. Nakashima, H. Zhang, X. Fujii, N. Uchiyama; T. Hattori, T. Marked increase in anti-HIV activity, as well as inhibitory activity against HIV entry mediated by CXCR4, linked to enhancement of the binding ability of tachyplesin analogs to CXCR4. AIDS Research and Human Retroviruses 15:419-427. 1999.
[18]. Bewley, C A. Louis, J M. Ghirlando, R. Clore G M. Design of a novel peptide inhibitor of HIV fusion that disrupts the internal trimeric coiled-coil of gp41. J. Biol. Chem. 277:14238-14245. 2002.
[19]. Ernst, J T. Kutzki, O. Debnath, A K. Jiang, S. Lu, H. Hamilton, A D. Design of a protein surface antagonist based on α-helix mimicry: inhibition of gp41 assembly and viral fusion. Angew. Chem. Int. Ed. 41:278-281. 2002.
[20]. Eckert, D M. Kim, P S. Design of potent inhibitors of HIV-1 entry from the gp41N-peptide region. Proc. Natl. Acad. Sci. USA. 98: 11187-11192. 2001.
[21]. Poveda, E. Rodes, B. Toro, C. Martin-Carbonero, L. Gonzalez-Lahoz, J. Soriano, V. Evolution of the gp41 env region in HIV-infected patients receiving T-20, a fusion inhibitor. AIDS 16:1959-61. 2002.
[22]. Su, S B. Gong, W H. Gao, J L. Shen, W P. Grimm, M C. Deng, X. Murphy, P M. Oppenheim, J J. Wang, J M. T20/DP178, an ectodomain peptide of human immunodeficiency virus type 1 gp41, is an activator of human phagocyte N-formyl peptide receptor. Blood 93:3885-92. 1999.
[23]. Neurath, A R. Strick, N. Li, Y Y. Anti-HIV-1 activity of anionic polymers: a comparative study of candidate microbicides. BMC Infections Diseases 2:27-38. 2002.
[24]. Kilby, J M. Hopkins, S. Venetta, T M. DiMassimo, B. Gretchen A. Cloud, G A. Lee, J Y. Alldredge, L. Hunter, E. Lambert, D. Bolognesi, D. Matthews, T. Johnson, M R. Nowak, M A. Shaw, G M. Saag, M S. Potent suppression of HIV-1 replication in humans by T-20, a peptide inhibitor of gp41-mediated virus entry. Nature Medicine 4:1302-1307. 1998.
[25]. Wild, C T. Shugars, D C. Greenwell, T K. McDanal, C B. Matthews, T J. Peptides corresponding to a predictive α-helical domain of human immunodeficiency virus type 1 gp41 are potent inhibitors of virus infection. Proc. Nat. Acad. Sci. USA. 91:9770-9774. 1994.
[26]. Sia, S K. Kim, P S. A designed protein with packing between left-handed and right-handed helices. Biochemistry 40:8981-8989. 2001.
[27]. Wild, C. Oas, T. McDanal, C. Bolognesi, D. Matthews, T. A synthetic peptide inhibitor of human immunodeficiency virus replication: correlation between solution structure and viral inhibition. Proc. Natl. Acad. Sci. USA. 89:10537-10541. 1992.
[28]. Siebert, X. Hummer, G. Hydrophobicity maps of the N-peptide coiled coil of HIV-1 gp41. Biochemistry 41:2956 -2961. 2002.
[29]. Wei, X. Decker, J M. Liu, H. Zhang, Z. Arani, R B. Kilby, J M. Saag, M S. Wu, X. Shaw, G M. Kappes, J C. Emergence of resistant human immunodeficiency virus type 1 in patients receiving fusion inhibitor (T-20) monotherapy. Antimicrobial Agents and Chemotherapy. 46:1896-1905. 2002.
[30]. Reeves, J D. Gallo, S A. Ahmad, N. Miamidian, J L. Harvey, P E. Sharron, M. Pöhlmann, S. Sfakianos, J N. Derdeyn, C A. Blumenthal, R. Hunter, E. Doms, R W. Sensitivity of HIV-1 to entry inhibitors correlates with envelope/coreceptor affinity, receptor density, and fusion kinetics. Proc. Natl. Acad. Sci. USA. 99:16249-16254. 2002.
[31]. Labrosse, B. Labernardiére, J-L. Dam, E. Trouplin, V. Skrabal, K. Clavel, F. Mammano, F. Baseline susceptibility of primary human immunodeficiency virus type 1 to entry inhibitors. J. Virol. 77:1610-1613. 2003.
[32]. Chan, D. Fass, D. Berger, J. Kim, P. Core structure of gp41 from the HIV envelope glycoprotein. Cell 89:263-273. 1997.
[33]. Tan, K. Liu, J-H. Wang, J-H. Shen, S. Lu, M. Atomic structure of a thermo stable subdomain of HIV-1 gp41. Proc. Natl. Acad. Sci. USA. 94:12303-8. 1997.
[34]. Kwong, P D. Wyatt, R. Robinson, J. Sweet, R W. Sodroski, J. Hendrickson, W A. Structure of an HIV gp120 envelope glycoprotein in complex with the CD4 receptor and a neutralizing human antibody. Nature 393:648-659. 1998.
[35]. Yang, J. Charles, M. Gabrys, C M. David P. Weliky, D P. Solid-state nuclear magnetic resonance evidence for an extended strand conformation of the membrane-bound HIV-1 fusion peptide. Biochemistry 40:8126 8137. 2001.
[36]. Louis, J M. Nesheiwat, I. Chang, L C. Clore, M. Carole A. Bewley, C A. Covalent trimers of the internal N-terminal trimeric coiled-coil of gp41 and antibodies directed against them are potent inhibitors of HIV envelope-mediated cell fusion. J. Biol. Chem. 2003.
[37]. Lawless, M K. Barney, S. Guthrie, K I. Bucy, T B. Petteway, S R. Jr. Merutka, G. HIV-1 membrane fusion mechanism: structural studies of the interactions between biologically active peptides from gp41. Biochemistry 35:13697-13708. 1996.
[38]. Lu, Y-A. Clavijo, P. Galantino, M. Shen, Z Y. Liu, W. Tam, J P. Chemically unambiguous peptide immunogen: preparation, orientation and antigenicity of purified peptide conjugated to the multiple antigen peptide system. Molecular Immunology 28:623-30. 1991.
[39]. Liu, C F. Tam, J P. Chemical ligation approach to form a peptide bond between unprotected peptide segments. Concept and model study. J. Am. Chem. Soc. 116:4149-53. 1994
[40]. Liu, C F. Tam, J P. Peptide segment ligation strategy without use of protecting groups. Proc. Natl. Acad. Sci. USA. 91:6584-48. 1994.
[41]. Tam, J. P. Miao, Z. Stereospecific pseudoproline ligation of N-terminal serine, threonine or cysteine. J. Am. Chem. Soc. 121:9013-9022. 1999.
[42]. Tam, J P. Yu, Q. Methionine ligation strategy in the biomimetic synthesis of parathyroid hormones. Biopolymers. 46:319-327. 1998.
[43]. Zhang, L. Torgerson, T. Liu, X-Y. Timmons, S. Colosia, A. Hawiger, J. Tam, J P. Preparation of functionally active cell-permeable peptides by single-step ligation of two peptide modules. Proc. Natl. Acad. Sci. USA. 95:9184-9189. 1998.
[44]. Zhang, L S. Tam, J P. Synthesis and application of unprotected cyclic peptides as building blocks for peptide dendrimers. J. Am. Chem. Soc. 119:2363-2370. 1997.
[45]. Li, X-F. Zhang, L-S. Hall, S E. Tam, J P. A new ligation method for N-terminal tryptophan-containing peptides using pictet-spengler reaction. Tetrahedron Lett. 41:4069-4073. 2000.
[46]. Tam, J P. Yu, Q. Miao, Z. Orthogonal ligation strategies for peptide and protein. Peptide Science 51, 311-332. 1999.
[47]. Tam, J P. Spetzler, J. Synthesis and application of peptide dendrimers as protein mimetics. Current Protocols in Protein Science, (Coligan, J. Dunn, B. Ploegh, H. Speicher, D. Wingfield, P. Eds.) 18.5.1-18.5.35. 1999.
[48]. Tam, J P. Xu, J. Eom, K D. Methods and strategies of peptide ligation. Biopolymers Peptide Science. 60:194-205. 2001.
[49]. Tam, J P. Yu, Q. A facile ligation approach to prepare three-helix bundles of HIV fusion-state protein mimetics. Organic Letters. 4:4167-70. 2002.
[50]. Eom, K D. Miao, Z. Yang, J-L. Tam, J P. Tandem ligation of multipartite peptide with cell permeable activity. J. Am. Chem. Soc. 125:73-82. 2003.
[51]. Sollner, T. Bennett, M K. Whiteheart, S W. Scheller, R H. Rothman, J E. A Protein assembly disassembly pathway in vitro that may correspond to sequential steps of synaptic vesicle docking, activation, and fusion. Cell 75:409. 1993.
[52]. Söllner, T. Rothman, J E. Neurotransmission: harnessing fusion machinery at the synapse. Trends in Neurosciences 17:344-348. 1994.
[53]. Chen, Y A. Scheller, R H. SNARE-mediated membrane fusion. Nature Reviews Molecular Cell Biology 2:98-106. 2001.
[54]. Eckert, D M. Malashkevich, V N. Hong, L H. Carr, P A. Kim, P S. Inhibiting HIV-1 entry: discovery of D-peptide inhibitors that target the gp41 coiled-coil pocket. Cell 99:103-105. 1999.
[55]. LaCasse, R A. Follis, K E. Trahey, M. Scarborough, J D. Littman, D R. Nunberg, J H. Fusion-competent vaccines: broad neutralization of primary isolates of HIV. Science 283:357-362. 1999.
[56]. Zhou, J. Aiken, C. Net enhances human immunodeficiency virus type-1 infectivity resulting from inter-virion fusion: evidence supporting a role for Net at the virion envelope. J. Virology 75:5851-5859. 2001.
[57]. Merrifield, R B. Solid phase peptide synthesis. Science 57:1619-1625. 1986.
[58]. Fields, G. Noble, R L. Solid phase peptide synthesis utilizing 9-fluorenylmethoxycarbonyl amino acids. Int. J. Peptide Protein Res. 35:161-214. 1990.
[59]. Shao, J. Tam, J P. Unprotected peptides as building blocks for the synthesis of peptide dendrimers with oxime, hydrazone, and thiazolidine linkages. J. Am. Chem. Soc. 117:3893-3899. 1995.
[60]. Rao, C. Tam, J P. Synthesis of peptide dendrimer. J. Am. Chem. Soc. 116:6975-6976. 1994.
[61]. Spetzler, J C. Tam, J P. Unprotected peptides as building blocks for branched peptides and peptide dendrimers. Intl. J. Peptide Protein Res. 45:78-85. 1995.
[62]. Otaka, A. Nakamura, M. Nameki, D. Kodama, E. Uchiyama, S. Nakamura, S. Nakano, H. Tamamura, H. Kobayashi, Y. Matsuoka, M. Fujii, N. Remodeling of gp41-C34 peptide leads to highly effective inhibitors of the fusion of HIV-1 with target cells. Angew. Chem. Int. Ed. 41:2936-2940. 2002.
[63]. Tam, J P. Yu, Q. Yang, J-L. Tandem ligation of unprotected peptides through thiopropyl and cysteinyl bonds in water. J. Am. Chem. Soc. 123:2487-2494. 2001.
[64]. Tam, J P. Yu, Q. Lu, Y-A. Tandem peptide ligation for synthetic and natural biologicals. Development in Biologicals. 29:189-96. 2001.
[65]. Miao, Z. Tam, J P. Bidirectional tandem pseudoproline ligations of proline-rich helical peptides. J. Am. Chem. Soc.122:4253-5260. 2000.
[66]. Chan, D C. Chutkowski, C T. Kim, P S. Evidence that a prominent cavity in the coiled coil of HIV type 1gp41 is an attractive drug target. Proc. Natl. Acad. Sci. USA. 95:15613-15617. 1998.
[67]. Cao, J. Bergeron, L. Helseth, E. Thali, M. Repke, H. Sodroski, J. Effects of amino acid changes in the extra cellular domain of the human immunodeficiency virus type I gp41 envelope glycoprotein. J. Virol. 67: 2747-2755. 1993.
[68]. Tam, J P. Lu, Y-A. Yang, J-L. Chiu, K.-W. An unusual structural motif of antimicrobial peptides containing end-to-end macrocycle and cysteine-knot disulfides. Proc. Natl. Acad. Sci. USA. 96:8913-8918. 1999.
[69]. Tam, J. P. Lu, Y-A. A biomimetic strategy in the synthesis and fragmentation of cyclic protein. Protein Science 7:1583-1592. 1998.
[70]. Tam, J P. Lu, Y-A. Yu, Q. Thia zip reaction for synthesis of large cyclic peptides: mechanisms and applications. J. Am. Chem. Soc. 121:4316-4324. 1999.
[71]. Jin, B S. Ryu, J R. Ahn, K. Yu, Y G. Design of a peptide inhibitor that blocks the cell fusion mediated by glycoprotein 41 of human immunodeficiency virus type 1. AIDS Research & Human Retroviruses. 16:1797-804. 2000.
[72]. Trkola, A. Paxton, W A. Monard, S P. Hoxie, J A. Siani, M A. Thompson, D A. Wu, L. Mackay, C R. Horuk, R. Moore, J P. Human immunodeficiency virus type 1 replication by CC and CXC chemokines. J. Virol. 72:396-404. 1998.
[73]. Ketas, T J. Frank, I. Klasse, P J. Sullivan, B M. Gardner, J P. Spenlehauer, C. Nesin, M. Olson, W C. Moore, J P. Pope, M. Human immunodeficiency virus type 1 attachment, coreceptor, and fusion Inhibitors are active against both direct and trans infection of primary cells. J. Virol. 77:2762-2767. 2003.
[74]. Rimsky, L T. Shugars, D C. Matthews, T J. Determinants of human immunodeficiency virus type 1 resistance to gp41-derived inhibitory peptides. J. Virol. 72:986-993. 1998.
[75]. Rosny, E. Vassell, R. Wingfield, P T. Wild, C T. Carol D. Weiss, C D. Peptides corresponding to the heptad-repeat motifs in the transmembrane protein (gp41) of human immunodeficiency virus type 1 elicit antibodies to receptor-activated conformations of the envelope glycoprotein. J. Virol. 75: 8859-8863. 2001.
[76]. Golding, H. Zaitseva, M. Rosny, E. King, L R. Manischewitz, J. Sidorov, I. Gorny, M K Zolla-Pazner, S. Dimitrov, D S. Weiss, C D. Dissection of human immunodeficiency virus type 1 entry with neutralizing antibodies to gp41 fusion intermediates. J. Virol. 76: 6780-6790. 2002.
[77]. He, Y. Vassell, R. Zaitseva, M. Nguyen, N. Yang, Z. Weng, Y. Weiss, C D. Peptides trap the human immunodeficiency virus type 1 envelope glycoprotein fusion intermediate at two sites. J. Virol. 77: 1666-1671. 2003.
[78]. DeGrado, W F. Schneider, J P. Hamuro, Y. The twists and turns of beta-peptides. J. Pep. Res. 54:206-217. 1999.
[79]. Liu, D H. DeGrado, W F. De novo design, synthesis, and characterization of antimicrobial β-peptides. J. Am. Chem. Soc. 123:7553-7559. 2001.
[80]. Seebach, D. Beck, A K. Rueping, M. Schreiber, J V. Sellner, H. Excursions of synthetic organic chemists to the world of oligomers andpolymers. Chimia. 55:98-103. 2001.
[81]. Wu, C S. Ikeda, K. Yang, J T. Ordered conformation of polypeptides and proteins in acid dodecyl sulfate solution. Biochemistry 20:566-570. 1981.
[82]. Biron, Z. Khare, S. Samson, A O. Hayek, Y. Naider, F. Anglister, J. A monomeric 3(10)-helix is formed in water by a 13-residue peptide representing the neutralizing determinant of HIV-1 on gp41. Biochemistry 41:12687 -12696. 2002.
[83]. Kliger, Y. Shai, Y. Inhibition of HIV-1 entry before gp41 folds into its fusion-active conformation. J. Mol. Bio. 295:163-168. 2000.
[84]. Lu M. Kim P S. A trimeric structural subdomain of the HIV-1 transmembrane glycoprotein. Journal of Biomolecular Structure & Dynamics. 15:465-71. 1997.
[85]. Macura, S. Ernst, R R. Elucidation of cross relaxation in liquids by two-dimensional NMR spectroscopy. Mol. Phys. 41:95-117. 1980.
[86]. Bothner-By, A A. Stephens, R L. Lee, J. Warren, C D. Jeanloz, R W. J. Am. Chem. Soc. Structure determination of a tetrasaccharide-transient nuclear overhauser effects in the rotating frame. J. Am. Chem. Soc. 106:811-813. 1984.
[87]. Rance M. Sorensen O W. Bodenhausen G. Wagner G. Ernst R R. Wuthrich K. Improved spectral resolution in cosy 1H NMR spectra of proteins via double quantum filtering. Biochem. Biophy. Res. Comm. 117:479-485. 1983.
[88]. Arepalli, S R. Glaudemans, C P. Daves, G D. Kovac, P. Bax, A. Identification of protein-mediated indirect NOE effects in a disaccharide-Fab′ complex by transferred ROESY. J. Magn. Res. Series B.106:195-198. 1995.
[89]. Barre-Sinoussi F, Chermann J C, Rey F, Nugeyre M T, Chamaret S, Gruest J, Dauguet C, Axler-Blin C, Vezinet-Brun F, Rouzioux C, Rozenbaum W, Montagnier L. Isolation of a T-lymphotropic retrovirus from a patient at risk for acquired immune deficiency syndrome (AIDS). Science 220:868-871. 1983.
[90]. Gallo R C, Salahuddin S Z, Popovic M, Shearer G M, Kaplan M, Haynes B F, Palker T J, Redfield R, Oleske J, Safai B, et al. Frequent detection and isolation of cytopathic retroviruses (HTLV-III) from patients with AIDS and at risk for AIDS. Science 224:500-503. 1984.
[91]. Clavel F, Guetard D, Brun-Vezinet F, Chamaret S, Rey M A, Santos-Ferreira M O, Laurent A G, Dauguet C, Katlama C, Rouzioux C, et al. Isolation of a new human retrovirus from West African patients with AIDS. Science 233:343-346. 1986.
[92]. Guyader M, Emerman M, Sonigo P, Clavel F, Montagnier L, Alizon M. Genome organization and transactivation of the human immunodeficiency virus type 2. Nature 326:662-669. 1987.
[93]. Varmus H. Retroviruses. Science 240:1427-1435. Review. 1988.
[94]. Hammarskjold M L, Rekosh D. The molecular biology of the human immunodeficiency virus. Biochim. Biophys. Acta. 989:269-280. Review. 1989.
[95]. Dalgleish A G, Beverley P C, Clapham P R, Crawford D H, Greaves M F, Weiss R A. The CD4 (T4) antigen is an essential component of the receptor for the AIDS retrovirus. Nature 312:763-767. 1984.
[96]. Maddon P J, Dalgleish A G, McDougal J S, Clapham P R, Weiss R A, Axel R. The T4 gene encodes the AIDS virus receptor and is expressed in the immune system and the brain. Cell 47:333-348. 1986.
[97]. McDougal J S, Kennedy M S, Sligh J M, Cort S P, Mawle A, Nicholson J K. Binding of HTLV-III/LAV to T4+ T cells by a complex of the 110K viral protein and the T4 molecule. Science 231:382-385. 1986.
[98]. Mitsuya H, Yarchoan R, Kageyama S, Broder S. Targeted therapy of human immunodeficiency virus-related disease. FASEB J. 5:2369-2381. 1991.
[99]. Mitsuya H, Yarchoan R, Broder S. Molecular targets for AIDS therapy. Science 249:1533-1544. Review 1990.
[100]. Larder B A, Darby G, Richman D D. HIV with reduced sensitivity to zidovudine (AZT) isolated during prolonged therapy. Science 243:1731-1734. 1989.
[101]. Erickson J, Neidhart D J, VanDrie J, Kempf D J, Wang X C, Norbeck D W, Plattner J J, Rittenhouse J W, Turon M, Wideburg N, et al. Design, activity, and 2.8 A crystal structure of a C₂ symmetric inhibitor complexed to HIV-1 protease. Science 249:527-533. 1990.
[102]. Barin F, McLane M F, Allan J S, Lee T H, Groopman J E, Essex M. Virus envelope protein of HTLV-III represents major target antigen for antibodies in AIDS patients. Science 228:1094-1096. 1985.
[103]. Smith D H, Byrn R A, Marsters S A, Gregory T, Groopman J E, Capon D J. Blocking of HIV-1 infectivity by a soluble, secreted form of the CD4 antigen. Science 238:1704-1707. 1987.
[104]. Schooley R T, Merigan T C, Gaut P, Hirsch M S, Holodniy M, Flynn T, Liu S, Byington R E, Henochowicz S, Gubish E, et al. Recombinant soluble CD4 therapy in patients with the acquired immunodeficiency syndrome (AIDS) and AIDS-related complex. A phase I-II escalating dosage trial. Ann Intern Med. 112:247-253. 1990.
[105]. Kahn J O, Allan J D, Hodges T L, Kaplan L D, Arri C J, Fitch H F, Izu A E, Mordenti J, Sherwin J E, Groopman J E, et al. The safety and pharmacokinetics of recombinant soluble CD4 (rCD4) in subjects with the acquired immunodeficiency syndrome (AIDS) and AIDS-related complex. A phase 1 study. Ann. Intern. Med. 112:254-261. 1990.
[106]. Ji H, Shu W, Burling F T, Jiang S, Lu M. Inhibition of human immunodeficiency virus type 1 infectivity by the gp41 core: role of a conserved hydrophobic cavity in membrane fusion. Journal of Virology 73:8578-8586. 1999.
[107]. Jiang S, Lin K, Strick N, Neurath A R. HIV-1 inhibition by a peptide. Nature 365:113. 1993.
[108]. Wild C, Greenwell T, Matthews T. A synthetic peptide from HIV-1 gp41 is a potent inhibitor of virus-mediated cell-cell fusion. AIDS Res. Hum. Retroviruses 9:1051-1053. 1993.
[109]. Wild C, Greenwell T, Shugars D, Rimsky-Clarke L, Matthews T. The inhibitory activity of an HIV type 1 peptide correlates with its ability to interact with a leucine zipper structure. AIDS Res. Hum. Retroviruses 11:323-325. 1995.
[110]. Wild C, Oas T, McDanal C, Bolognesi D, Matthews T. A synthetic peptide inhibitor of human immunodeficiency virus replication: correlation between solution structure and viral inhibition. Proc. Natl. Acad. Sci. USA 89:10537-10541. 1992.
[111]. Wild C T, Shugars D C, Greenwell T K, McDanal C B, Matthews T J. Peptides corresponding to a predictive alpha-helical domain of human immunodeficiency virus type 1 gp41 are potent inhibitors of virus infection. Proc. Natl. Acad. Sci. USA 91:9770-9774. 1994.
[112]. Chan D C, Chutkowski C T, Kim P S. Evidence that a prominent cavity in the coiled coil of HIV type 1 gp41 is an attractive drug target. Proc. Natl. Acad. Sci. USA 95:15613-15617. 1998.
[113]. Ferrer M, Kapoor T M, Strassmaier T et al. Selection of gp41-mediated HIV-1 cell entry inhibitors from biased combinatorial libraries of non-natural binding elements. Nat. Struct. Biol. 6:953-960. 1999.
[114]. Eckert D M, Malashkevich V N, Hong L H, Carr P A, Kim P S. Inhibiting HIV-1 entry: discovery of D-peptide inhibitors that target the gp41 coiled-coil pocket. Cell 99:103-115. 1999.
[115]. Kilby J M, Hopkins S, Venetta T M et al. Potent suppression of HIV-1 replication in humans by T-20, a peptide inhibitor of gp41-mediated virus entry. Nat. Med. 4:1302-1307. 1998.
[116]. Weiss R. L., Teich N., Varmus H. and Coffin, J. (Eds.); RNA tumor viruses. 2^{nd} Edition, 2:1070-1085; Cold Spring Harbor Laboratory, Cold Spring Harbor 1985.

1. A protein mimetic for preventing HIV-1 entry to host cells of a living subject through membrane fusion, wherein HIV-1 contains at least one envelope glycoprotein gp41 that has a plurality of peptides in a pre-hairpin state, comprising:
a. at least two monomeric peptide strands; and
b. an interstrand linker coupling the monomeric peptide strands.

2. The protein mimetic of claim 1, wherein the coupled monomeric peptide strands prevent the plurality of trimeric gp41 in a pre-hairpin state from transiting to a six-helix hairpin bundle, thereby inhibiting HIV-1 entry to the host cells through membrane fusion.

3. The protein mimetic of claim 1, wherein each of the two strands has an amino acid sequence, which contains at least one of N36, DP178, T1249, C34, any other amino acid sequences derived from N-peptide or C-peptide regions of gp41, or any truncated, mutated, modified linear or cyclized analogs there of.

4. The protein mimetic of claim 1, wherein the at least two monomeric peptide strands can be the same or chimeric.

5. The protein mimetic of claim 1, wherein the at least two monomeric peptide strands are coupled by the interstrand linker through a chemical, enzymatic, or biological synthetic method.

6. The protein mimetic of claim 5, wherein the chemical synthetic method includes chemoselective thiazolidine ligation, Trp ligation, ψGly ligation, Michael addition ligation, disulfide linkage, or any combination there of.

7. The protein mimetic of claim 1, wherein the interstrand linker has at least two arms represented by formula 1 or 2: 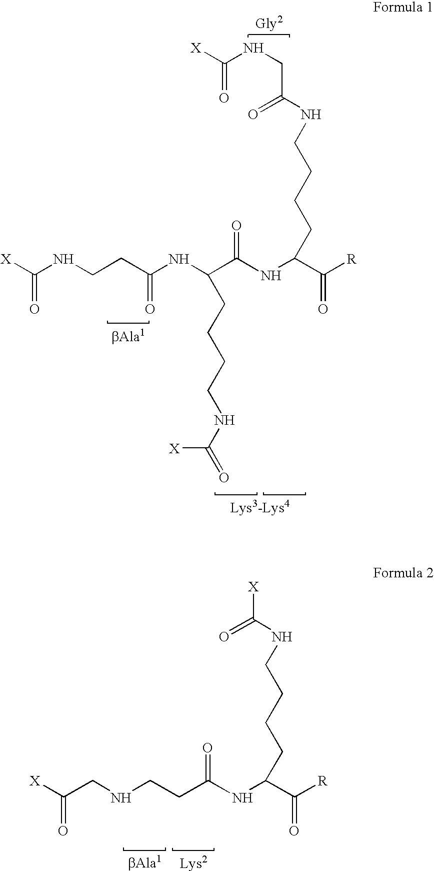
wherein X can be an aldehyde, β-aminoethyl thiol, chloroacetyl or acrylate, and R is Ser-Ser-Ala-NH₂.

8. A pharmaceutical composition suitable for administration to a living subject for preventing or treating infections caused by HIV-1 viral entry to host cells of the living subject through membrane fusion, wherein HIV-1 contains at least one envelope glycoprotein gp41 that has a plurality of peptides in a pre-hairpin state, comprising a pharmaceutically acceptable protein mimetic having:
a. at least two monomeric peptide strands; and
b. an interstrand linker coupling the monomeric peptide strands.

9. The pharmaceutical composition of claim 8, further comprising a pharmaceutically acceptable carrier suitable for administration to a living subject.

10. The pharmaceutical composition of claim 8, wherein the coupled monomeric peptide strands prevent the plurality of trimeric gp41 in a pre-hairpin state from transiting to a six-helix hairpin bundle, thereby inhibiting HIV-1 entry to the host cells through membrane fusion.

11. The pharmaceutical composition of claim 8, wherein each of the two strands has an amino acid sequence, which contains at least one of N36, DP178, T1249, C34, any other amino acid sequences derived from N-peptide or C-peptide regions of gp41, or any truncated, mutated, modified linear or cyclic analogs there of.

12. The pharmaceutical composition of claim 8, wherein the at least two monomeric peptide strands can be the same or chimeric.

13. The pharmaceutical composition of claim 8, wherein the at least two monomeric peptide strands are coupled by the interstrand linker through a chemical, enzymatic, or biological synthetic method.

14. The pharmaceutical composition of claim 13, wherein the chemical synthetic method includes chemoselective thiazolidine ligation, Trp ligation, ψGly ligation, Michael addition ligation, disulfide linkage, or any combination there of.

15. The pharmaceutical composition of claim 8, wherein the interstrand linker has at least two arms represented by formula 1 or 2: 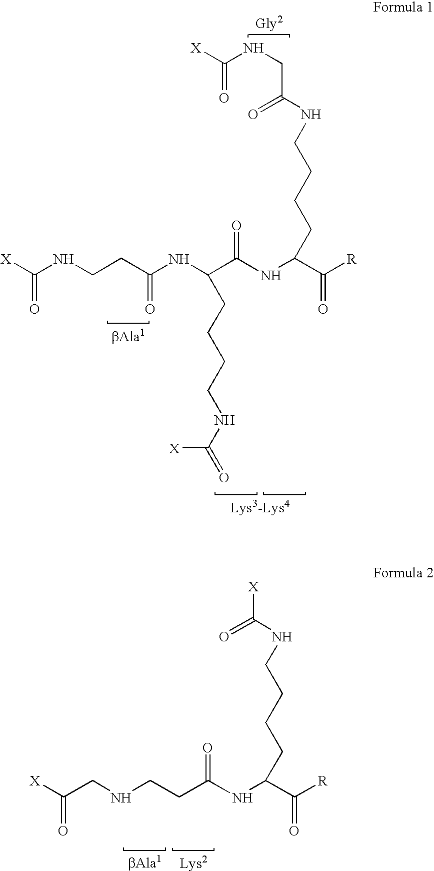
wherein X can be an aldehyde, β-aminoethyl thiol, chloroacetyl or acrylate, and R is Ser-Ser-Ala-NH₂.

16. A therapeutic or prophylactic method against HIV-1 infection by inhibiting viral entry to host cells of a living subject through membrane fusion, wherein HIV-1 contains at least one envelope glycoprotein gp41 that has a plurality of peptides in a pre-hairpin state, comprising administering to a living subject an effective amount of a protein mimetic, wherein the protein mimetic comprises:
a. at least two monomeric peptide strands; and
b. an interstrand linker coupling the monomeric peptide strands.

17. The method of claim 16, wherein the coupled monomeric peptide strands prevent the plurality of trimeric gp41 in a pre-hairpin state from transiting to a six-helix hairpin bundle, thereby inhibiting HIV-1 entry to the host cells through membrane fusion.

18. The method of claim 16, wherein each of the two strands has an amino acid sequence, which contains at least one of N36, DPI78, T1249, C34, any other amino acid sequences derived from N-peptide or C-peptide regions of gp41, or any truncated, mutated, modified linear or cyclic analogs there of.

19. The method of claim 16, wherein the at least two monomeric peptide strands can be the same or chimeric.

20. The method of claim 16, wherein the at least two monomeric peptide strands are coupled by the interstrand linker through a chemical, enzymatic, or biological synthetic method.

21. The method of claim 20, wherein the chemical synthetic method includes chemoselective thiazolidine ligation, Trp ligation, ψGly ligation, Michael addition ligation, disulfide linkage, or any combination there of.

22. The method of claim 16, wherein the interstrand linker has at least two arms represented by formula 1 or 2: 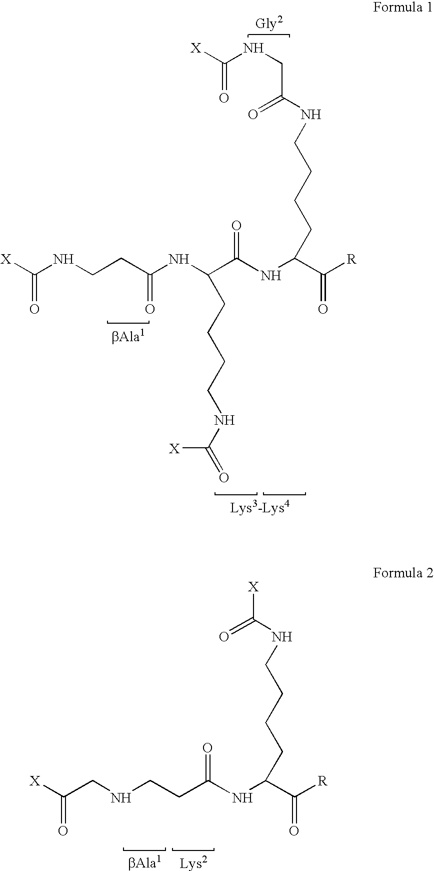
wherein X can be an aldehyde, β-aminoethyl thiol, chloroacetyl or acrylate, and R is Ser-Ser-Ala-NH₂.

23. A protein mimetic for preventing viral entry of a virus to host cells of a living subject through membrane fusion, wherein the virus contains at least one protein that has a plurality of peptides in a pre-hairpin state, comprising:
a. at least two monomeric peptide strands; and
b. an interstrand linker coupling the monomeric peptide strands.

24. The protein mimetic of claim 23, wherein the coupled monomeric peptide strands prevent the plurality of peptides of the protein in a pre-hairpin state from transiting to a hairpin bundle, thereby inhibiting viral entry to the host cells through membrane fusion.

25. The protein mimetic of claim 23, wherein the virus is one of HIV-1, Ebola, influenza, SARS-coV, retroviruses, corona viruses, orthomyxoviruses or paramyxoviruses.

26. The protein mimetic of claim 23, wherein each of the two strands has an amino acid sequence, which is derived from N-peptide or C-peptide regions of the protein, or any truncated, mutated, modified linear or cyclic analogs there of.

27. The protein mimetic of claim 23, wherein the at least two monomeric peptide strands can be the same or chimeric.

28. The protein mimetic of claim 23, wherein the at least two monomeric peptide strands are coupled by the interstrand linker through a chemical, enzymatic, or biological synthetic method.

29. The protein mimetic of claim 23, wherein the interstrand linker has at least two arms represented by formula 1 or 2: 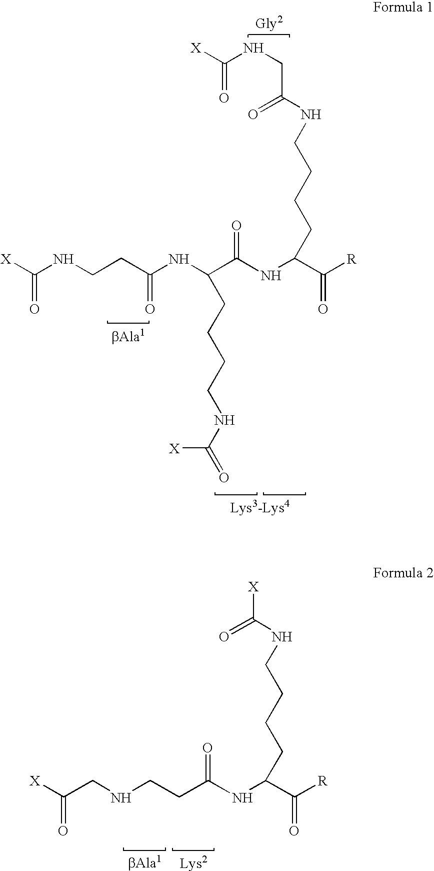
wherein X can be an aldehyde, β-aminoethyl thiol, chloroacetyl or acrylate, and R is Ser-Ser-Ala-NH₂.

30. A pharmaceutical composition suitable for administration to a living subject for preventing or treating infections caused by viral entry of a virus to host cells of the living subject through membrane fusion, wherein the virus contains at least one protein that has a plurality of peptides in a pre-hairpin state, comprising a pharmaceutically acceptable protein mimetic having:
a. at least two monomeric peptide strands; and
b. an interstrand linker coupling the monomeric peptide strands.

31. The pharmaceutical composition of claim 30, further comprising a pharmaceutically acceptable carrier suitable for administration to a living subject.

32. The pharmaceutical composition of claim 30, wherein the coupled monomeric peptide strands prevent the plurality of peptides of the protein in a pre-hairpin state from transiting to a hairpin bundle, thereby inhibiting viral entry to the host cells through membrane fusion.

33. The pharmaceutical composition of claim 30, wherein the virus is one of HIV-1, Ebola, influenza, SARS-coV, retroviruses, corona viruses, orthomyxoviruses or paramyxoviruses.

34. The pharmaceutical composition of claim 30, wherein each of the two strands has an amino acid sequence, which is derived from N-peptide or C-peptide regions of the protein, or any truncated, mutated, modified linear or cyclic analogs there of.

35. The pharmaceutical composition of claim 30, wherein the at least two monomeric peptide strands can be the same or chimeric.

36. The pharmaceutical composition of claim 30, wherein the at least two monomeric peptide strands are coupled by the interstrand linker through a chemical, enzymatic, or biological synthetic method.

37. The pharmaceutical composition of claim 30, wherein the interstrand linker has at least two arms represented by formula 1 or 2: 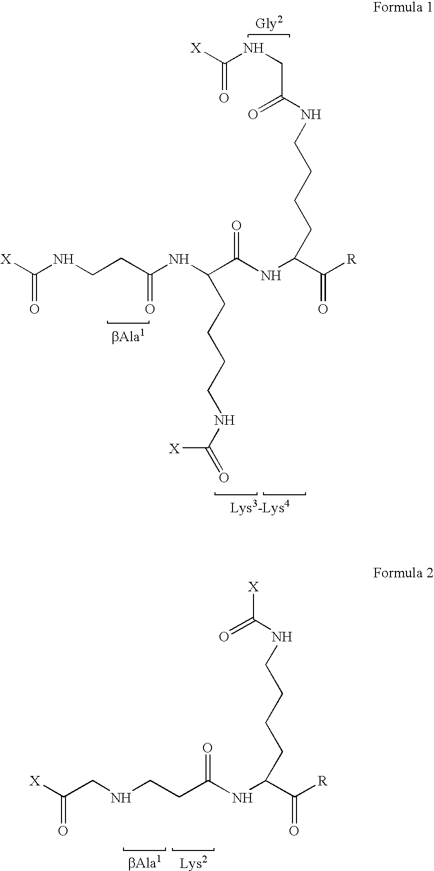
wherein X can be an aldehyde, β-aminoethyl thiol, chloroacetyl or acrylate, and R is Ser-Ser-Ala-NH₂.

38. A therapeutic or prophylactic method against viral infection by inhibiting viral entry of a virus to host cells of a living subject through membrane fusion, wherein the virus contains at least one protein that has a plurality of peptides in a pre-hairpin state, comprising administering to a living subject an effective amount of a protein mimetic, wherein the protein mimetic comprises:
a. at least two monomeric peptide strands; and
b. an interstrand linker coupling the monomeric peptide strands.

39. The method of claim 38, wherein the coupled monomeric peptide strands prevent the plurality of peptides of the protein in a pre-hairpin state from transiting to a hairpin bundle, thereby inhibiting viral entry to the host cells through membrane fusion.

40. The protein mimetic of claim 38, wherein the virus is one of HIV-1, Ebola, influenza, SARS-coV, retroviruses, corona viruses, orthomyxoviruses or paramyxoviruses.

41. The method of claim 38, wherein each of the two strands has an amino acid sequence, which is derived from N-peptide or C-peptide regions of the protein, or any truncated, mutated, modified linear or cyclic analogs there of.

42. The method of claim 38, wherein the at least two monomeric peptide strands can be the same or chimeric.

43. The method of claim 38, wherein the at least two monomeric peptide strands are coupled by the interstrand linker through a chemical, enzymatic, or biological synthetic method.

44. The method of claim 38, wherein the interstrand linker has at least two arms represented by formula 1 or 2: 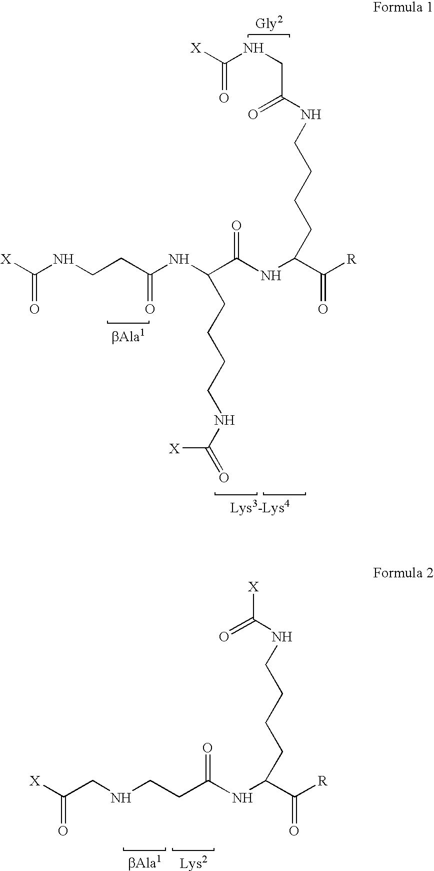
wherein X can be an aldehyde, β-aminoethyl thiol, chloroacetyl or acrylate, and R is Ser-Ser-Ala-NH₂.

45. A protein mimetic for inhibiting membrane fusion, wherein the membrane contains at least one protein that has a plurality of peptides in a pre-hairpin state, comprising:
a. at least two monomeric peptide strands; and
b. an interstrand linker coupling the monomeric peptide strands.

46. The protein mimetic of claim 45, wherein the coupled monomeric peptide strands prevent the plurality of peptides of the protein in a pre-hairpin state from transiting to a hairpin bundle, thereby inhibiting membrane fusion.

47. The protein mimetic of claim 45, wherein the membrane fusion can be vesicle fusion or any membrane fusion event that involves a hairpin mediated step.

48. The protein mimetic of claim 45, wherein each of the two strands has an amino acid sequence, which is derived from N-peptide or C-peptide regions of the protein, or any truncated, mutated, modified linear or cyclic analogs there of.

49. The protein mimetic of claim 45, wherein the at least two monomeric peptide strands can be the same or chimeric.

50. The protein mimetic of claim 45, wherein the at least two monomeric peptide strands are coupled by the interstrand linker through a chemical, enzymatic, or biological synthetic method.

51. The protein mimetic of claim 45, wherein the interstrand linker has at least two arms represented by formula 1 or 2: 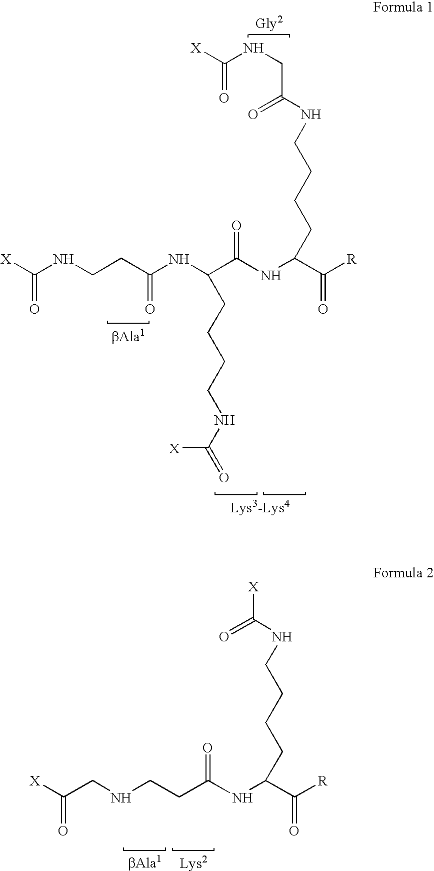
wherein X can be an aldehyde, β-aminoethyl thiol, chloroacetyl or acrylate, and R is Ser-Ser-Ala-NH₂.


SEQUENCE LISTING (8 sequences)

SEQ ID NO: 1 - protein, 36 aa, Human immunodeficiency virus type 1
SGIVQQQNNLLRAIEAQQHLLQLTVWGIKQLQARIL

SEQ ID NO: 2 - protein, 36 aa, Human immunodeficiency virus type 1
YTSLIHSLIEESQNQQEKNEQELLELDKWASLWNWF

SEQ ID NO: 3 - protein, 40 aa, Human immunodeficiency virus type 1
WMEWYTSLIHSLIEESQNQQEKNEQELLELDKWASLWNWF

SEQ ID NO: 4 - protein, 34 aa, Human immunodeficiency virus type 1
WMEWDREINNYTSLIHSLIEESQNQQEKNEQELL

SEQ ID NO: 5 - protein, 27 aa, Human immunodeficiency virus type 1
INNYTSLIHSLIEESQNQQEKNEQELL

SEQ ID NO: 6 - protein, 16 aa, Human immunodeficiency virus type 1
IEESQNQQEKNEQELL

SEQ ID NO: 7 - protein, 24 aa, Human immunodeficiency virus type 1
QNQQEKNEQELLELDKWASLWNWF

SEQ ID NO: 8 - protein, 13 aa, Human immunodeficiency virus type 1
LELDKWASLWNWF